(19) United States
(12) Patent Application Publication
(10) Pub. No.: US 2010/0040623 A1
(43) Pub. Date: Feb. 18, 2010

(54) NEURITOGENIC AND NEURONAL SURVIVAL PROMOTING PEPTIDES DERIVED FROM THE FAMILY OF S-100 PROTEINS

(76) Inventors: Elisabeth Bock, Charlottenlund (DK); Vladimir Berezin, Copenhagen (DK); Darya Kiryushko, Copenhagen (DK)

Correspondence Address: GIFFORD, KRASS, SPRINKLE,ANDERSON & CITKOWSKI, P.C, PO BOX 7021, TROY, MI, 48007-7021, US

(21) Appl. No.: 12/442,177

(22) PCT Filed: Dec. 20, 2006
(86) PCT No.: PCT/DK2006/000730

§ 371 Date: Mar. 20, 2009

(30) Foreign Application Priority Data
Dec. 20, 2005 (DK) PA 2005 01796

(51) Int. Cl. A61K 39/395; C07K 7/06; C07K 7/08; C07K 14/00; A61K 38/08; A61K 38/10; A61K 38/16; C07K 16/00

(52) U.S. Cl. 424/139.1; 530/324; 530/325; 530/326; 530/327; 530/328; 530/329; 514/17; 514/16; 514/15; 514/14; 514/13; 514/12; 530/387.9

(57) ABSTRACT

The present invention relates to peptide fragments derived from proteins of the S-100 family promoting neural cell survival, differentiation and proliferation. The invention further relates to pharmaceutical compositions comprising said peptide fragments and uses thereof for treatment of diseases and conditions where the effects of stimulating neural cell proliferation, differentiation and/or survival, and/or stimulating neural plasticity associated with learning and memory are beneficial for treatment.

### FIELD OF INVENTION

The present invention relates to neural cell survival differentiation and proliferation promoting peptide fragments derived from proteins of the S-100 family, pharmaceutical compositions comprising said peptide fragments and uses thereof for treatment of diseases and conditions where the effects of stimulating neural cell proliferation, differentiation and/or survival, and/or stimulating neural plasticity associated with learning and memory are beneficial for treatment.

### BACKGROUND OF INVENTION

The S100 family is a group of vertebrate-specific Ca2+-binding proteins with a highly conserved primary structure possessing both intra- and extracellular functions. Most S100 family members, including S100A4, are antiparallelly packed homodimers stabilized by disulphide bridges (Donato, 2001). Intracellularly, S100 proteins are involved in a variety of processes including regulation of cytoskeletal dynamics, Ca2+ homeostasis, and cell proliferation and differentiation. Importantly, some S100 proteins can also be secreted, form oligomers owing to the nonreducing conditions of the environment, and exert their effects acting at the cell surface (Kerkhoff et al., 1998). A plasma membrane target for S100B and S100A12, the receptor for advanced glycation end products (RAGE), has been identified on inflammatory and neural cells (Hofmann et al., 1999). However, RAGE is probably not the sole receptor for members of the S100 family, since effects of extracellular S100A12 and S100B proteins can be observed in cells lacking RAGE (Robinson et al., 2002), and some of these effects are RAGE-independent in cells expressing the receptor (Sorci et al., 2002).

The gene of S100A4 (also termed Mts1) was isolated from tumor cells (Ebralidze et al., 1989), where its expression increased the ability of the tumor to metastasise. S100A4 has also been detected in normal tissues, in particular, in the nervous system. Both in brain and spinal cord, S100A4 expression appears in astrocytes shortly after the start of myelination, with the highest level observed in the areas, in which neurogenesis takes place, and in regions possessing high plasticity in the adult (Åberg and Kozlova, 2000). Moreover, in the peripheral nervous system, expression of S100A4 increases after sciatic nerve or dorsal root injury (Kozlova and Lukanidin, 1999). Thus, the release of S100A4 from S100A4-positive astrocytes as a result of either secretion or cell damage might play a role in neuronal plasticity under normal and pathological conditions. Importance of S100A4 in brain development and/or regeneration is supported by the fact that the protein is a potent promoter of neurite outgrowth in hippocampal neurons in vitro (Novitskaya et al., 2000). Moreover, S100A4 acts as a neuroprotectant for primary neurons induced to undergo cell death (Pedersen et al., 2004). The molecular mechanism of this effect, including a receptor transducing S100A4 signals, has not been identified. However, the S100A4-induced neurite outgrowth could be reduced by inhibitors of intracellular Ca2+ homeostasis (Novitskaya et al., 2000). This indicates that extracellular S100A4 might affect the intracellular Ca2+ concentration ([Ca2+]i) thereby modulating neuronal differentiation.

The crystal structure of human EF-hand calcium-binding protein S100A12 in its calcium-bound form has been determined to 1.95 A resolution by molecular replacement using the structure of the S100B protein (Moroz et al., 2001). Like the majority of S100 proteins, S100A12 is a dimer, with the interface between the two subunits being composed mostly of hydrophobic residues. The fold of S100A12 is similar to the other known crystal and solution structures of S100 proteins, except for the linker region between the two EF-hand motifs. Sequence and structure comparison between members of the S100 family suggests that the target-binding region in S100A12 is formed by the linker region and C-terminal residues of one subunit and the N-terminal residues of another subunit of the dimer. The N-terminal region of the target-binding site includes two glutamates that are conserved in most of the S100 sequences. The precise role of S100A12 in cell behaviour is yet undefined, as is the case for the whole family, although it has been shown that the interaction of S100A12 with the RAGE receptor is implicated in inflammatory response (Hofmann et al., 1999). Human recombinant S100A12 has been found to dramatically induce neuritogenesis of hippocampal neurons (Mikkelsen et al., 2001). The response to S100A12 was dependent on the dose in a bell-shaped manner. A 10-fold increase in neurite outgrowth was observed upon treatment with S100A12 in concentrations between 0.1 and 2.0 μm already after 24 h. Exposure to S100A12 for only 15 min was enough to induce neuritogenesis when measured after 24 h, but to obtain a maximal response, S100A12 had to be present in the culture for at least 4 h. The response to S100A12 is abolished by inhibitors of phospholipase C (PLC), protein kinase C (PKC), Ca²⁺ flux, Ca²⁺/calmodulin dependent kinase II (CaMKII) or mitogen-activated protein kinase (MEK).

### REFERENCES

Bain J R, Mackinnon S E, Hudson A R, Falk J A, and D A Hunter 1988. The peripheral nerve allograft: an assessment of regeneration across nerve allografts in rats immunosuppressed with cyclosporin A. Plast Reconstr Surg. 82(6):1052-66.
Donato R 2001. S100: A multigenic family of calcium-modulated proteins of the EFhand type with intracellular and extracellular functional roles. Int J Biochem Cell Biol 33:637-668.
Ebralidze A, Tulchinsky E, Grigorian M, Afanasyeva A, Senin V, Revazova E, and E Lukanidin 1989. Isolation and characterization of a gene specifically expressed in different metastatic cells and whose deduced gene product has a high degree of homology to a Ca2+-binding protein family. Genes Dev 3:1086-1093.
Hofmann M A, Drury S, Fu C, Qu W, Taguchi A, Lu Y, Avila C, Kambham N, Bierhaus A, Nawroth P, Neurath M F, Slattery T, Beach D, McClary J, Nagashima M, Morser J, Stern D, and A M Schmidt 1999. RAGE mediates a novel proinflammatory axis: a central cell surface receptor for S100/calgranulin polypeptides Cell 97:889-901.
Kerkhoff C, Klempt V, and C Sorg 1998. Novel insights into structure and function of VRP8 S100A8 and MRP14 S100A9. Biochim Biophys Acta 1448:200-211.
Kozlova E N and E Lukanidin 1999. Metastasis-associated Mts1/S100A4 protein is selectively expressed in white matter astrocytes and is upregulated after peripheral nerve or dorsal root injury. Glia 27:249-258.
Mikkelsen S E, Novitskaya V, Kriajevska M, Berezin V, Bock E, Norrild B, and E. Lukanidin 2001. S100A12 protein is a strong inducer of neurite outgrowth from primary hippocampal neurons. J. Neurochem. 79:767-776.
Moroz O V, Antson A A, Murshudov G N, Maitland N J, Dodson G G, Wilson K S, Skibshoj I, Lukanidin E M, Bronstein I B. 2001. The three-dimensional structure of human S100A12. Acta Crystallogr D Biol Crystallogr 57:20-29.
Novitskaya V, Grigorian M, Kriajevska M, Tarabykina S, Bronstein I, Berezin V, Bock E, and E Lukanidin 2000. Oligomeric forms of the metastasis-related Mts1/S100A4 protein stimulate neuronal differentiation in cultures of rat hippocampal neurons. J Biol Chem 52:41278-41286.
Pedersen M, Kohler L, Grigorian M, Novitskaya V, Bock E, Lukanidin E, Berezin V 2004. The Mts1/S100A4 protein is a neuroprotectant. J Neurosci Res 77:777-86.
Penkowa M, Giralt M, Lago N, Camats J, Carrasco J, Hernandez J, Molinero A, Campbell I L, Hidalgo J. 2003. Astrocyte-targeted expression of IL-6 protects the CNS against a focal brain injury. Exp Neurol. 181:1301-48.
Robinson M. J, Tessier P, Poulsom R, and N Hogg 2002. The S100 family heterodimer, MRP-8/14, binds with high affinity to heparin and heparan sulphate glycosaminoglycans on endothelial cells. J Biol Chem 277:3658-3665.
Rønn L C, Olsen M, Ostergaard S, Kiselyov V, Berezin V, Mortensen M T, Lerche M H, Jensen P H, Soroka V, Saffell J L, Doherty P, Poulsen F M, Bock E, Holm A. 1999. Identification of a neuritogenic ligand of the neural cell adhesion molecule using a combinatorial library of synthetic peptides. Nat Biotech 17:1000-1005.
Rønn L C, Ralets I, Hartz B P, Bech M, Berezin A, Berezin V, Moller A, Bock E. 2000. A simple procedure for quantification of neurite outgrowth based on stereological principles. J Neurosci Methods, 100:25-32.
Sorci G, Riuzzi F, Marchetti C, and R Donato 2002. S100B causes apoptosis in myoblasts and inhibits myogenic differentiation and myotube formation in a RAGE-independent manner. Seventh European Symposium on Calcium-Binding Proteins in Normal and Transformed Cells, Brussels, Belgium, Jun. 12-16, 2002, PJ42.
Åberg F, and E Kozlova 2000. Metastasis-associated Mts1/S100A4 protein in the developing and adult central nervous system. J Comp Neurol 424:269-282.

### SUMMARY OF THE INVENTION

The present invention relates to peptide sequences capable of stimulating neuronal cell differentiation, neuronal cell survival and neural plasticity associated with learning and memory and capable of inhibiting of inflammatory response. According to the invention, the peptide sequences described herein comprise a common structural motif which confers on the peptides biological activity.

Thus, in the first aspect the present invention relates to a peptide comprising a sequence of at most 30 contiguous amino acid residues comprising an amino acid motif of the formula:

x^{p}-(x1)-(x2)-(x3)-x^{p}-(x4),
wherein
x^{P} is a hydrophobic amino acid residue
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

In another aspect the invention relates to a compound comprising a peptide sequence comprising the amino acid motif of above.

A peptide sequence of the invention is preferably derived for a protein of the S100 family. The invention discloses particular examples of such peptide sequences. These sequences comprise the motif disclosed herein and are capable of stimulating neuronal cell differentiation, neuronal cell survival and/or neural plasticity associated with learning and memory.

The invention also relates to a pharmaceutical composition comprising a peptide sequence of the invention and/or compound comprising thereof.

Further aspects of the invention include a method of stimulating neural cell survival, differentiation, proliferation and/or plasticity associated with memory and learning comprising using a peptide sequence according to the invention and/or a pharmaceutical composition comprising thereof.

Then, the invention relates to uses of disclosed peptide sequences and compounds comprising thereof for the manufacture of a medicament and/or for the production of an antibody.

The antibodies of the invention are capable binding to an epitope comprising the structural motif described herein or a peptide sequence disclosed herein. The invention also relates to a pharmaceutical composition comprising an antibody of the invention.

The invention also concerns methods of treatment of individuals in need comprising administering a peptide sequence, compound or antibody or a pharmaceutical composition comprising thereof.

### DESCRIPTION OF THE DRAWINGS

FIG. 1: Design of S100-derived peptides. The sequences of S100A4 and S100A12 proteins were divided into six regions each, and the tetrameric peptides representing individual regions were generated. The peptides were termed hekatones (H) 1 to 6. Thereafter, all twelve peptides were tested for their neuritogenic activity in primary neurons.

FIG. 2: Identification of the neurite inducing regions in sequences of S100A4 and S100A12. The S100A4- and S100A12-derived peptides (H1-H6, see FIG. 1 for details) were tested for their neuritogenic activity in primary hippocampal neurons (upper panel, HN) and cerebellar neurons (lower panel, CN). Two neuritogenic regions have been identified in the sequence of S100A4 corresponding to the H3 and H6 peptides, and one region has been determined in the S100A12 sequence corresponding to the H3 peptide. The H3 and H6 peptides derived from S100A4 were further characterized in vitro, and are henceforth referred to as H3 and H6. *p<0.05, **p<0.01, ***p<0.001 when compared to the untreated controls.

FIG. 3: Identification of aminoacids responsible for the neuritogenic effect of the H3 and H6 peptides (upper and lower panels, respectively). First, truncated versions of the peptides were tested for their ability to induce neurite outgrowth from primary hippocampal neurons. Non-truncated versions were used as controls. It was found that only the C-terminal part of the H3 peptide was crucial for the induction of neurite outgrowth (upper panel, left), whereas for the H6 peptides, truncation of just two aminoacids from the N-terminal resulted in the loss of neuritogenic activity (lower panel, left). Based on truncation experiments, a minimal sequence required for the induction of neurite outgrowth was identified for each peptide (shown underlined on top of respective truncation graphs). These sequences were subsequently used for the alascan experiments, where individual amino acids were substituted by alanins in order to identify residues crucial for the neuritogenic activities of H3 and H6 (upper and lower panels, respectively, right). We have found four amino acids in each peptide sequence, which were important for the induction of neurite outgrowth by H3 and H6 (shown underlined on top of respective alascan graphs). (*)-statistical significance P<0.05, (**)-p<0.01, (***)-p<0.001, Anova analysis.

FIG. 4: H3 and H6 activate intracellular messengers mediating neurite outgrowth and survival of primary neurons. Primary hippocampal neurons cultured for 6 hours were stimulated with H3 or H6 for 15 minutes. Thereafter, an activity (phosphorylation level) of the three intracellular messengers involved in neurite outgrowth and survival, Akt, CREB, and ERK, was determined using the PAGE method. Both peptides significantly increased phosphorylation of all three messengers. (*)-statistical significance P<0.05, (**)—P<0.01, (***)—P<0.001, Anova analysis.

FIG. 5: S100A4-derived peptides (H3, H6) increase neuronal survival. Primary cerebellar neurons cultured for 7 days in high potassium medium ([KCl]=40 mM) were induced to undergo apoptosis by lowering of KCl concentration to 5 mM. Both H3 and H6 peptides partially rescued cerebellar neurons increasing the survival by 30-40%. (*)-statistical significance P<0.05, (**)—P<0.01, (***)—P<0.001, Anova analysis.

FIG. 6: H6, but not H3 increases intracellular Ca²⁺ concentration ([Ca²+]ᵢ). Since Ca²⁺ has been demonstrated to play a crucial role in the processes of neurite outgrowth and survival, and S100A4 is known to increase [Ca²⁺]ᵢ in primary neurons, we loaded cultured hippocampal neurons with a ratiometric Ca²⁺-sensitive dye fura-2 and tested whether the S100A4-derived peptides affected [Ca²⁺]ᵢ. It has been found that application of H6, but not H3 significantly increased the level of intracellular Ca²⁺. (*)-statistical significance P<0.05, (**)—P<0.01, (***)—P<0.001, Anova analysis.

FIG. 7: H3 and H6 protect the brain from the kainic acid (KA)-induced toxicity. Both H3 and H6 decreased amount of seizures induced by 20 mg (kg KA (top). Moreover, the peptides shifted the profile of seizures induced by 30 mg/kg KA towards less severe seizures types and decreased the mortality by 30-50% (bottom). (*)-statistical significance P<0.05, (**)—P<0.01, (***)—P<0.001, Anova analysis.

FIG. 8: The H3 peptide accelerates functional recovery after sciatic nerve crush in rats. Wistar rats have been subjected to a sciatic nerve crush at day 0. Thereafter, rats were divided into three groups, each group receiving daily injections of either vehicle (Veh), or the H3 and H6 peptides at a dose of 10 mg/kg for 18 days. Every second day, the Sciatic Function Index (SFI) was evaluated in all groups based on the analysis of walking tracks of individual animals (SFI=−100 corresponding to a complete limb paralysis, SFI=0 corresponding to a normal limb function). It has been found that the H3 peptide strongly promoted the recovery of the motor function of the injured limb. (*)-statistical significance p<0.05, (**)—P<0.01, (***)—P<0.001, Anova analysis.

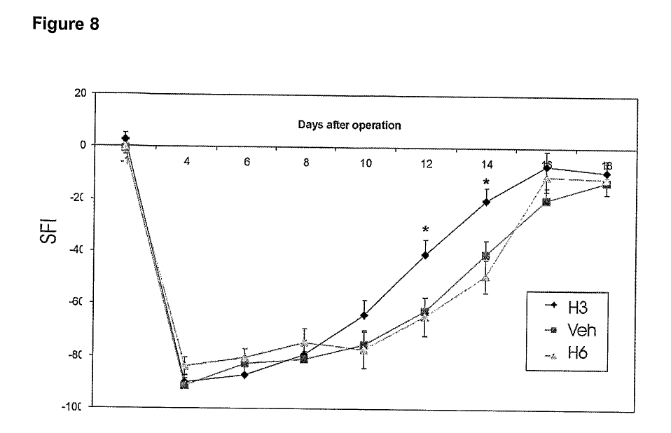

FIG. 9: Effect of peptides derived from the S100A4 proteins on DNA-fragmentation at post-lesion day 4. The peptides were administered subcutaneously in a dose of 10 mg/kg at day (−)1, 1 and 2. Tunel stained cells are indicated by arrows.

FIG. 10: Quantification of the effect of peptides derived from the S100A4 proteins on DNA-fragmentation at post-lesion day 4. The peptides were administered subcutaneously in a dose of 10 mg/kg at day (−)1, 1 and 2. *p<0.05, **p<0.01 when compared to the vehicle treated control.

FIG. 11: Effect of peptides derived from the S100A4 protein on the number of GFAP-positive cells at post-lesion day 4. The peptides were administered subcutaneously in a dose of 10 mg/kg at day (−)1, 1 and 2. **p<0.01 when compared to the vehicle-treated controls.

FIG. 12: Effect of peptides derived from the S100A4 protein on the area of GFAP-immunoreactivity (% of the total penumbra area). The peptides were administered subcutaneously in a dose of 10 mg/kg at day (−)1, 1 and 2. ***p<0,001 when compared to the vehicle-treated controls.

FIG. 13: Effect of peptides derived from the S100A4 protein on the number of proliferating cells at post-lesion day 4. The peptides were administered subcutaneously in a dose of 10 mg/kg at day (−)1.1 and 2. *p<0.05 when compared to the vehicle-treated controls.

### DETAILED DESCRIPTION OF THE INVENTION

### 1 Peptide Sequences

### Individual Peptide Sequences

First aspect of the invention relates to a peptide comprising at most 30 contiguous amino acid residues comprising an amino acid motif of the formula:

x^{p1}-(x1)-(x2)-(x3)-x^{p2}-(x4),
wherein
x^{p1} and x^{p2} are hydrophobic amino acid residues,
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

Hydrophobic amino acid residues x^{p1} and x^{p2} of the motif may be selected from any hydrophobic residues, however in some embodiments residues L, V, I, M, F or A may be preferred.

Amino acid residues (x1), (x2) and (x3) may be any amino acid residues with the proviso that at least one of (x1), (x2) or (x3) is an amino acid residue selected from E, D, K, R, Q, N, S or T. In some embodiments it may be preferred that any two of (x1), (x2) or (x3) are residues selected from E, D, K, R, Q, N, S or T. In some other embodiments it may be preferred that any one of (x1), (x2) or (x3) residues is an amino acid residue selected from E, D, K, R, Q, N, S or T, any another of (x1), (x2) or (x3) residues is a hydrophobic amino acid residue and the third residue is any amino acid residue.

In one preferred embodiment of the invention the motif of above comprises at least two contiguous hydrophobic residues. The hydrophobic residues may be any hydrophobic residues selected from A, F, I, L, M, P, V or W. In some further preferred embodiments the two contiguous hydrophobic residues may be preceded or followed by a hydrophilic residue selected from E, D, K, R, Q, N, S or T. In other further preferred embodiments the two contiguous hydrophobic residues may be preceded or followed by a hydrophobic residue. The contiguous hydrophobic residues may be in one embodiment the residues x^{p2} and (x4), in another embodiment the residues (x3) and x^{p2}, in still another embodiments the residues x^{p1} and (x1) or residues (x1) and (x2).

In one preferred embodiment the sequence (x3)-x^{p2}-(x4) of the motif comprises (x3) which is an amino acid residue selected from E, D, K, R, Q, N, S or T and (x4) which is a hydrophobic amino acid residue. The latter hydrophobic residue in one embodiment may be preferably selected from residues P or A, in another embodiment preferable selected from residues I or L, and in still another embodiment preferably selected from residues F or M. In another preferred embodiment (x4) may be an amino acid residue selected from E, D, K, R, Q, N, S or T and (x3) is any hydrophobic amino acid residue. The hydrophobic residue (x3) in this case may be selected from V, L, I, A or F.

A peptide sequence comprising a motif according to invention may comprise from 6 to 50 contiguous amino acid residues.

In one embodiment, the sequence may be of about 50 amino acid residues in length, such as for example from 40 to 49 amino acid resides. In another embodiment the sequence may comprise less then 40 amino acid residues, for example from 30 to 39 amino acid residues, such as less then 30 amino acid residues, for example from 20 to 29, such as about 25 amino acid residues. The sequence may also be less then 20 amino acid residues, such as from 10 to 19 amino acid residues, for example from 10 to 15 amino acid residues, for example such as 10, 11, 12, 13, 14 or 15 amino acid residues, or for example from 16 to 19 amino acid residues, such as 16, 17, 18 or 19 amino acid residues. Peptides comprising less then 10 amino acid residues are also in the scope, such as for example 6, 7, 8 or 9 amino acid residues. In particular, the invention concerns the peptides which length is in the range of 6 to 15 amino acid residues or in the range of 10 to 25 amino acid residues.

A peptide comprising the above described motif may, in particular, comprise a sequence selected from the amino acid sequences identified herein as SEQ ID NOs: 1-85 or consists of any of the sequences SEQ ID NOs:1-85, such as for example SEQ ID NOs:1-5. A sequence selected from SEQ ID NOs:1-5 is a preferred peptide sequence of the invention.

In another preferred embodiment a peptide of the invention may comprise or consist of a sequence selected from SEQ ID NOs:51-68.

In still another preferred embodiment the peptide may comprise or consists of a sequence selected from SEQ ID NOs:6-22.

In yet another preferred embodiment the peptide may comprise or consist of a sequence selected from SEQ ID NOs:23-42.

In further preferred embodiments the peptide may comprise or consists of a sequence selected from SEQ ID NOs:43-50, or a sequence selected from SEQ ID NOs:52-68, or a sequence selected from SEQ ID NOs:69-85.

In more preferred embodiments the invention concern the sequences of SEQ ID NOs: 1-5.

In one preferred embodiment the peptide may comprise or consists of sequence NEFFEGFPDKQPRKK (SEQ ID NO: 1), or comprise a fragment or variant of sequence NEFFEGFPDKQPRKK (SEQ ID NO:1). In another preferred embodiment, the peptide may comprise or and consists of sequence KELLTRELPSFLGKRT (SEQ ID NO:2), or comprise a fragment or variant of said sequence, in still another preferred embodiment it may comprise or consists of sequence KQLLTKELANTIKNIK (SEQ ID NO: 3), or comprise a fragment or variant thereof. Still, in another preferred embodiments the peptide may comprise or and consists of sequence DEAAFQKLMSNLD (SEQ ID NO: 4) or sequence CPLEKALDVMVSTF (SEQ ID NO:5), or comprise a fragment or variant of any of these sequences.

In the present content the fragment is defined as an amino acid sequence comprising at least 5 contiguous amino acid residues of a sequence selected from SEQ ID NOs:1-85, such as SEQ ID NOs:1-5, said amino acid sequences comprising two contiguous hydrophobic preceding or following a residue selected from K, R, D, E, N, Q, S or T.

The variant in the present content is defined as an amino acid sequence of 6 to 50 contiguous amino acid residues, which has at least 50% sequence similarity with a sequence selected from the sequences of SEQ ID NOs:1-85, preferably, more then 50% sequence similarity with a sequence selected from sequences of SEQ ID NOs:1-85, such as for example from 51% to 60%, preferably more then 60%, for example between 61% and 70%, more preferably more then 70% sequence similarity, such as from 71% to 80%-85%, more preferred sequence similarity about 90%, such as from 85% to 90%, and even more preferred sequence similarity about 99%. In another embodiment, a variant of a sequence selected from SEQ ID NOs:1-85 may be a sequence of SEQ ID NOs:1-85 which comprises modifications of amino acid residues discussed below. Other variants of peptide sequences of the invention concerned are also discussed below.

Sequence similarity/homology/identity may be may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90. The terms “sequence similarity” sequence identity” and “sequence homology” are used in the present application interchangeably when referred to a number or percentage of identical or similar amino acid residues in two collated amino acid sequences. “Similar amino acid residues” are amino acid residues derived from the same group of “conservative” amino acid residues. The latter groups are discussed further in the application.

Both, the fragments and variants as above are according to the invention functional equivalents of peptide sequences of SEQ ID NOs:1-85.

In the present application the standard one-letter code for amino acid residues is applied as well as the standard three-letter code. Abbreviations for amino acids are in accordance with the recommendations in the IUPAC-IUB Joint Commission on Biochemical Nomenclature Eur. J. Biochem, 1984, vol. 184, pp 9-37. Throughout the description and claims either the three letter code or the one letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a peptide of the invention exists as the free carboxylic acid, this may also be specified as “—OH”. However, the C-terminal amino acid of a compound of the invention may be the amidated derivative, which is indicated as “—NH₂”. Where nothing else is stated the N-terminal amino acid of a polypeptide comprise a free amino-group, this may also be specified as “H—”.

Where nothing else is specified amino acid can be selected from any amino acid, whether naturally occurring or not, such as alfa amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His Aib, NaI, Sar, Orn, Lysine analogues, DAP, DAPA and 4Hyp.

Also, according to the invention modifications of the compounds/peptides may be performed, such as for example glycosylation and/or acetylation of the amino acids. Basic amino acid residues are according to invention represented by the residues of amino acids Arg, Lys, and His, acidic amino acid residues—by the residues of amino acids Glu and Asp. Basic and acidic amino acid residues constitute a group of charged amino acid residues. The group of hydrophobic amino acid residues is represented by the residues of amino acids Leu, Ile, Val, Phe, Trp, Tyr, Met, Ala and Pro.

The invention relates to naturally occurring, synthetically/recombinant prepared peptide sequence/fragments, and/or peptide sequence/fragments prepared by means of enzymatic/chemical cleavage of a bigger polypeptide, wherein said peptide sequence/fragments are integral parts of said bigger polypeptides. The invention relates to isolated individual peptide sequences.

As mentioned above, the invention also relates to variants of peptide sequences described above.

In one aspect the term “variant of a peptide sequence” means that the peptides may be modified, for example by substitution of one or more of the amino acid residues. Both L-amino acids and D-amino acids may be used. Other modification may comprise derivatives such as esters, sugars, etc. Examples are methyl and acetyl esters.

In another aspect “variants” may be understood as exhibiting amino acid sequences gradually differing from the preferred predetermined sequence, as the number and scope of insertions, deletions and substitutions including conservative substitutions increase. This difference is measured as a reduction in homology between the predetermined sequence and the variant.

In still another aspect, variants of the peptide fragments according to the invention may comprise, within the same variant, or fragments thereof or among different variants, or fragments thereof, at least one substitution, such as a plurality of substitutions introduced independently of one another. Variants of the complex, or fragments thereof may thus comprise conservative substitutions independently of one another, wherein at least one glycine (Gly) of said variant, or fragments thereof is substituted with an amino acid selected from the group of amino acids consisting of Ala, Val, Leu, and Ile, and independently thereof, variants, or fragments thereof, wherein at least one alanine (Ala) of said variants, or fragments thereof is substituted with an amino acid selected from the group of amino acids consisting of Gly, Val, Leu, and Ile, and independently thereof, variants, or fragments thereof, wherein at least one valine (Val) of said variant, or fragments thereof is substituted with an amino acid selected from the group of amino acids consisting of Gly, Ala, Leu, and Ile, and independently thereof, variants, or fragments thereof, wherein at least one leucine (Leu) of said variant, or fragments thereof is substituted with an amino acid selected from the group of amino acids consisting of Gly, Ala, Val, and Ile, and independently thereof, variants, or fragments thereof, wherein at least one isoleucine (Ile) of said variants, or fragments thereof is substituted with an amino acid selected from the group of amino acids consisting of Gly, Ala, Val and Leu, and independently thereof, variants, or fragments thereof wherein at least one aspartic acids (Asp) of said variant, or fragments thereof is substituted with an amino acid selected from the group of amino acids consisting of Glu, Asn, and Gln, and independently thereof, variants, or fragments thereof, wherein at least one aspargine (Asn) of said variants, or fragments thereof is substituted with an amino acid selected from the group of amino acids consisting of Asp, Glu, and Gln, and independently thereof, variants, or fragments thereof, wherein at least one glutamine (Gln) of said variants, or fragments thereof is substituted with an amino acid selected from the group of amino acids consisting of Asp, Glu, and Asn, and wherein at least one phenylalanine (Phe) of said variants, or fragments thereof is substituted with an amino acid selected from the group of amino acids consisting of Tyr, Trp, His, Pro, and preferably selected from the group of amino acids consisting of Tyr and Trp, and independently thereof, variants, or fragments thereof, wherein at least one tyrosine (Tyr) of said variants, or fragments thereof is substituted with an amino acid selected from the group of amino acids consisting of Phe, Trp, His, Pro, preferably an amino acid selected from the group of amino acids consisting of Phe and Trp, and independently thereof, variants, or fragments thereof, wherein at least one arginine (Arg) of said fragment is substituted with an amino acid selected from the group of amino acids consisting of Lys and His, and independently thereof, variants, or fragments thereof, wherein at least one lysine (Lys) of said variants, or fragments thereof is substituted with an amino acid selected from the group of amino acids consisting of Arg and His, and independently thereof, variants, or fragments thereof, and independently thereof, variants, or fragments thereof, and wherein at least one proline (Pro) of said variants, or fragments thereof is substituted with an amino acid selected from the group of amino acids consisting of Phe, Tyr, Trp, and His, and independently thereof, variants, or fragments thereof, wherein at least one cysteine (Cys) of said variants, or fragments thereof is substituted with an amino acid selected from the group of amino acids consisting of Asp, Glu, Lys, Arg, His, Asn, Gln, Ser, Thr, and Tyr.

It thus follows from the above that the same functional equivalent of a peptide fragment, or fragment of said functional equivalent may comprise more than one conservative amino acid substitution from more than one group of conservative amino acids as defined herein above. The term “conservative amino acid substitution” is used synonymously herein with the term “homologous amino acid substitution”.

The groups of conservative amino acids are as the following:

P, A, G (neutral, weakly hydrophobic),
S, T (neutral, hydrophilic)
Q, N (hydrophilic, acid amine)
E, D (hydrophilic, acidic)
H, K, R (hydrophilic, basic)
L, I, V, M, F, Y, W (hydrophobic, aromatic)
C (cross-link forming)

Conservative substitutions may be introduced in any position of a preferred predetermined peptide of the invention or fragment thereof. It may however also be desirable to introduce non-conservative substitutions, particularly, but not limited to, a non-conservative substitution in any one or more positions.

A non-conservative substitution leading to the formation of a functionally equivalent fragment of the peptide of the invention would for example differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on peptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

As it was mentioned above the present invention relates to fragments and variants of the peptide sequences described above. The following fragments and variants are preferred by the invention.

A fragment which is a sequence which is about 40%, more preferably at least 50%, more preferably at least 60%, more preferably at least 70%, more preferably at least 80%, more preferably at least 90%, more preferably at least 95% of the length of a sequence selected from the sequences of SEQ ID NOs: 1-85. It may be preferred a fragment that comprises at least 6 amino acid residues and the motif x^{p}-(x1)-(x2)(x3)-x^{p}-(x4), wherein x^{p} is a hydrophobic amino acid residue, at least one of (x1), (x2) or (x3) is an amino acid residue selected from E, D, K, R, Q, N, S or T, and (x4) is an amino acid residue selected from E, D, K, R, Q, N, S or T or a hydrophobic amino acid residue. It may also preferred a fragment which comprises at least 5 contiguous amino acid residues of a sequence of SEQ ID NOs:1-85, comprising two contiguous hydrophobic amino acid residues preceded or followed by a residues selected from E, D, K, R, Q, N, S or T.

A variant which is an amino acid sequence having at least 60%, more preferably at least 70%, more preferably at least 80%, more preferably at least 90%, more preferably 95% homology to a sequence comprising the motif of the invention, in particular to a sequence selected from the sequences of SEQ ID NOs: 1-85, or is an amino acid sequence having at least 60%, more preferably at least 70%, more preferably at least 80%, more preferably at least 90%, more preferably 95% positive amino acid matches compared to a sequence comprising the motif of the invention, in particular to a sequence of SEQ ID NOS: 1-85. A positive amino acid match is defined herein as an identity or similarity defined by physical and/or chemical properties of the amino acids having the same position in two compared sequences. Preferred positive amino acid matches of the present invention are K to R, E to D, L to M, Q to E, I to V, I to L, A to S, Y to W, K to Q, S to T, N to S and Q to R. The homology of one amino acid sequence with another amino acid is defined as a percentage of identical amino acids in the two collated sequences. The homology of the sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90;

The preferred fragments and variants of the invention functional homologues/equivalents of sequences identified as SEQ ID NOs:1-85, which means that the fragments and variants has at least some biological activity of the original sequence, for example a capability of stimulating neural plasticity, such as associated with neural cell differentiation and/or such as associated with memory and learning, stimulating of cell survival, such as inhibiting apoptosis, and/or activating a receptor.

As mentioned above, the invention relates both to naturally occurring, synthetically or recombinantly prepared peptides and peptides prepared by means of enzymatic/chemical cleavage of a bigger polypeptide sequence. The peptides produced by enzymatic cleavage of a bigger polypeptide sequence, as well as peptides, which are prepared by means of recombinant expression or by means of chemical synthesis, wherein said peptide sequences are corresponding to integral sequences of bigger polypeptides or proteins, are according to invention derived from the sequences of said bigger polypeptides or proteins.

The invention preferably relates to peptides which are derived from the sequences of the proteins belonging to the S100 family of proteins, in particular, S100A1 (Swiss-prot Ass. number: P23297), S100A2 (Swiss-prot Ass. number: P29034), S100A3 (Swiss-prot Ass. number: P33764), S100A4 (Swiss-prot Ass. number: P26447), S100A5 (Swiss-prot Ass. number: P33763), S100A6 (Swiss-prot Ass. number: P06703), S100A7 (Swiss-prot Ass. number: P31151), S100A8 (Swiss-prot Ass. number: P05109), S100A9 (Swiss-prot Ass. number: P06702), S100A10 (Swiss-prot Ass. number: P60903), S100A11 (Swiss-prot Ass. number: P31949), S100A12 (Swiss-prot Ass. number: P80511), S100A13 (Swiss-prot Ass. number: Q99584), S100A14 (Swiss-prot Ass. number: Q9HCY8), S100A16 (Swiss-prot Ass. number: Q96FQ6), S100B (Swiss-prot Ass. number: P04271), S100G (Swiss-prot Ass. number: P29377), S100P (Swiss-prot Ass. number: P25815) and S100Z (Swiss-prot Ass. number: Q8WXG8).

In one preferred embodiment a peptide may consists of or comprise a sequence corresponding to a fragment of sequence of S100A4. Such peptide according to the invention is derived from S100A4 protein. Examples of interesting peptides which are derived from S100A4 protein represented in the application by SEQ ID NOs: 4, 23-42 and 81.

### 2. Compound

A compound may contain a single copy of an individual amino acid sequence selected from any of the described above, or it may contain two or more copies of such amino acid sequence. This means that compound of the invention may be formulated as a monomer of a peptide sequence, such as containing a single individual peptide sequence, or it may be formulated as a multimer of a peptide sequence, i.e containing two or more individual peptide sequences, wherein said individual peptide sequences may be represented by two or more copies of the same sequence or by two or more different individual peptide sequences. A multimer may also comprises a combination of the full-length sequence and one or more fragments thereof. In one embodiment a compound may contain two amino acid sequences, such compound is defined herein as dimer, in another embodiment a compound may contain more then two amino acid sequences, such for example three, four or more sequences. The present invention preferably relates to compounds containing two or four peptide sequences of the invention. However, compounds containing 3, 5, 6, 7, 8 or more sequences are also in the scope of the invention.

The compounds may be formulated as dimers or multimers comprising more then two copies of individual peptide fragments which may have the identical amino acid sequences or different amino acid sequences. One example of such compound may be a dimeric compound containing SEQ ID NO: 1 and SEQ ID NO: 2 or a dimeric compound containing SEQ ID NO: 1 and SEQ ID NO: 3. Any other combinations of the sequences of the invention may be made depending on different embodiments. The sequences may be connected to each other via peptide bond, or connected to each other through a linker molecule or grouping.

As already mentioned above, a compound of the invention may contain two or more copies of a single sequence, such as for example two copies of any of the sequences selected from SEQ ID NOs: 1-85, wherein said two sequences may be connected to each other via a linker molecule or grouping. A compound wherein the sequences are connected via a linker grouping is preferred. One example of such linking grouping may be an achiral di-, tri- or tetracarboxylic acid. Suitable achiral di-, tri- or tetracarboxylic acids and a method of production such a compound (a ligand presentation assembly method (LPA)) are described in WO0018791 and WO2005014623. Another example of a possible linker may be the amino acid lysine. Individual peptide sequences may be attached to a core molecule such as lysine forming thereby a dendritic multimer (dendrimer) of an individual peptide sequence(s). Production of dendrimers is also well known in the art (PCT/US90/02039, Lu et al., (1991) Mol. Immunol. 28:623-630; Defoort et al., (1992) Int J Pept Prot Res. 40:214-221; Drijfhout et al. (1991) Int J Pept Prot Res. 37:27-32), and dedrimers are at present widely used in research and in medical applications. It is a preferred embodiment of the invention to provide a dendrimeric compound comprising four individual amino acid sequences attached to the lysine core molecule. It is also preferred that at least one of the four individual amino acid sequences comprises an amino acid sequence of the formula defined above. It is even more preferred if the all four individual amino acid sequences of a dendrimeric compound individually comprise an amino acid sequence of the formula defined above.

Multimeric compounds of the invention, such as LPA-dimers or Lysin-dendrmers, are preferred compounds of the invention. However, other types of multimeric compounds comprising two or more individual sequences of the invention may be preferred depending on the embodiments.

### 3. Biological Activity

A peptide sequence of the invention and a compound comprising a sequence of the invention possess biological activity. The invention preferably relates to a biological activity selected from
capability of stimulating stem cell proliferation and/or differentiation, for example neural cell precursor cell proliferation and/or differentiation;
capability of stimulating neural cell differentiation, for example stimulating neurite outgrowth or regeneration of nerves;
capability neural plasticity associated with memory and learning, for example stimulating synaptic efficacy;
capability of stimulating of cell survival, for example inhibiting apotosis,
capability of inhibiting inflammation, such as stimulating anti-inflammatory response;
capability of binding to a receptor, for example Sphingosine 1-phosphate receptor Edg-3 (Swiss-prot Ass. number: Q99500), and modulating activity of said receptor, such as stimulating or inhibiting signal transduction associated with this receptor;
capability of modulating cell motility, such as inhibiting cellular migration and cancer cell dissemination;
capability of regulating cell differentiation, such stimulating or inhibiting differentiation of endothelial cells.

Thus, according to one embodiment of the invention a peptide sequence of the invention is capable of stimulating neuronal cell differentiation.

The term “neuronal differentiation” is understood herein both as differentiation of neural precursor cells, or neural stem cells, and further differentiation of neural cells, such as for example maturation of neuronal cells. An example of such differentiation may be neurite outgrowth from immature neurons, branching of neurites, and also neuron regeneration.

Thus, one preferred embodiment the invention concerns biological activity of a peptide sequence associated with stimulating of differentiation of neural precursor/stem cells or immature neurons, in another preferred embodiment the invention concern stimulating neurite outgrowth from mature neurons, for examples neurons which were traumatizes but survived and are committed to regenerate damaged processes. Accordingly, the invention also concerns a method for stimulating neuronal cell differentiation comprising using a peptide sequence of the invention or a compound comprising said sequence.

Substances with the potential to promote neurite outgrowth as well as stimulate regeneration and/or differentiation of neuronal cells, such as certain endogenous trophic factors, are prime targets in the search for compounds that facilitate for example neuronal regeneration and other forms of neuronal plasticity. To evaluate the potential of the present compound, the ability to stimulate the neurite outgrowth related signalling, interfere with cell adhesion, stimulate neurite outgrowth, regeneration of nerves, may be investigated. Compounds of the present invention are shown to promote neurite outgrowth and are therefore considered to be good promoters of regeneration of neuronal connections, and thereby of functional recovery after damages as well as promoters of neuronal function in other conditions where such effect is required.

In the present context “differentiation” is related to the processes of maturation of neurons and extension of neurites, which take place after the last cell division of said neurons. The compounds of the present invention may be capable of stopping neural cell division and initiating maturation said cells, such as initiating extension of neurites. Otherwise, “differentiation” is related to initiation of the process of genetic, biochemical, morphological and physiological transformation of neuronal progenitor cells, immature neural cells or embryonic stem cells leading to formation of cells having functional characteristics of normal neuronal cell as such characteristics are defined in the art. The invention defines “immature neural cell” as a cell that has at least one feature of neural cell accepted in the art as a feature characteristic for the neural cell.

According to the present invention a compound comprising at least one of the above peptide sequences is capable of stimulating neurite outgrowth. The invention concerns the neurite outgrowth improvement/stimulation such as about 75% improvement/stimulation above the value of neurite outgrowth of control/non-stimulated cells, for example 50%, such as about 150%, for example 100%, such as about 250, for example 200%, such as about 350%, for example 300%, such as about 450%, for example 400%, such as about 500%.

Estimation of capability of a candidate compound to stimulate neurite outgrowth may be done by using any known method or assay for estimation of neurite outgrowth, such as for example as the described in Examples below.

According to the invention a compound has neuritogenic activity both as an insoluble immobile component of cell growth substrate and as a soluble component of cell growth media. In the present context “immobile” means that the compound is bound/attached to a substance which is insoluble in water or a water solution and thereby it becomes insoluble in such solution as well. For medical applications both insoluble and soluble compounds are considered by the application, however soluble compounds are preferred. Under “soluble compound” is understood a compound, which is soluble in water or a water solution.

One of most preferred embodiments of the invention concerns the activity of the peptide sequences in connection with learning and memory, in particular, the capability of a peptide sequence to stimulate synaptic plasticity, spine formation, synaptic efficacy. Thus, the invention also concerns a method for stimulating memory and/or learning comprising using a peptide sequence of the invention and/or compound comprising said sequence. The invention relates to both shortterm memory and long-term memory.

In another preferred embodiment of the invention a peptide sequence of the invention capable of stimulating cell survival, in particular neuronal cell survival. The invention concerns the capability of stimulating cell survival both due trauma and degenerative disease. Accordingly, the invention relates to a method for stimulating cell survival, preferably neuronal cell survival by using a peptide sequence of the invention and/or compound comprising said sequence.

Substances with the potential to enhance neuronal cells to survive due to damage as well as inhibit degeneration and/or apoptosis of neuronal cells in trauma and disease, are prime targets in the search for candidate compounds for new medicine for treatment of neurodegenerative diseases such as for example Alzheimer's or Parkinson's diseases. To evaluate the potential of the present peptides, the ability to stimulate survival related signalling, interfere with apoptosis related cellular reactions, stimulate regeneration of nerves may be investigated. Compounds of the present invention are shown to promote neural cell survival and decrease the cell loss and therefore considered to be good candidates for promotion of regeneration of neural connections in brain and/or in peripheral neural system, and thereby of functional recovery after damages due trauma or disease as well as promoters of neuronal function in any other conditions where such effect is required.

In the present context “survival” is related to the processes associated with maintenance and/or recovery of cell function after the damage of the cell. The compounds of the present invention may be capable of stopping or attenuating the processes committing the cell to death, such as inhibiting apoptosis of neural cells initiated by cell damage due trauma or disease. Otherwise, “survival” is related to inhibition of the processes associated with the cell damage leading to cell death and initiation of the processes of genetic, biochemical, morphological and physiological transformation or reconstruction of cells, in particular neuronal cells, such as progenitor cells, immature neural cells or embryonic stem cells or mature neural cells having normal functional characteristics defined in the art. The invention defines “immature neural cell” as a cell that has at least one feature of neural cell accepted in the art as a feature characteristic for the neural cell.

According to the present invention a compound comprising at least one of the above peptide sequences is capable of stimulating neural cell survival. The invention concerns the neural cell survival stimulation such as about 75% stimulation above the value of survival of control/non-stimulated cells, for example 50%, such as about 150%, for example 100%, such as about 250, for example 200%, such as about 350%, for example 300%, such as about 450%, for example 400%, such as about 500%.

Estimation of capability of a candidate compound to stimulate neural cell survival may be done by using any known method or assay for estimation of cell survival, such as for example the ones described in Examples of the present application.

According to the invention a compound has survival promoting activity both as insoluble and soluble compound. In the present context “insoluble” means that the compound is bound/attached to a substance which is insoluble in water or a water solution and thereby the compound becomes insoluble in such solution as well. For medical applications both insoluble and soluble compounds are considered by the application, however soluble compounds are preferred. Under “soluble compound” is understood a compound, which is soluble in water or a water solution.

In another embodiment the peptide sequence of the invention is also capable of inhibiting an inflammatory process, in particular an inflammatory process in the brain.

Inflammation is a defense reaction caused by tissue damage due to a mechanical injury or bacterial, virus or other organism infection. The inflammatory response involves three major stages: first, dilation of capillaries to increase blood flow; second, microvascular structural changes and escape of plasma proteins from the bloodstream; and third, leukocyte transmigration through endothelium and accumulation at the site of injury and infection. The inflammatory response begins with a release of inflammatory mediators. Inflammatory mediators are soluble, diffusible molecules that act locally at the site of tissue damage and infection, and at more distant sites, influencing consequent events of the inflammatory response. Inflammatory mediators can be exogenous, e.g. bacterial products or toxins, or endogenous, which are produced within the immune system itself, as well as injured tissue cells, lymphocytes, mast cells and blood proteins.

Neuroinflammation plays a prominent role in the progression of Alzheimer's disease and may be responsible for degeneration in vulnerable regions such as the hippocampus. Neuroinflammation is associated with elevated levels of extracellular glutamate and potentially an enhanced stimulation of glutamate N-methyl-D-aspartate receptors.

Anti-inflammatory is another important biological activity of the peptide sequence of the invention. Thus, the invention relates to anti-inflammatory peptide, which is capable of serving as an inhibitor of the sustained inflammatory response, in particular in the brain.

The continuous presence of inflammatory mediators, such as for example TNF alpha in the body in response to sustained presence of bacterial products or even live bacteria locally during days or weeks following trauma and/or infection promotes the reactions to inflammation, such as, for example, heat, swelling, and pain. The sustained inflammatory response has been proven to be very harmful to the body. If the bacterial products or live bacteria become spread universally in the body from their local focus the inflammatory reaction becomes overwhelming and out of control and leads to sepsis which eventually progress further to severe sepsis and septic shock. Anti-inflammatory peptides may be used to block or suppress the overwhelming sustained inflammatory response represented by a massive and harmful cytokine cascade in the blood and vital organs such as lung, liver intestine, brain and kidneys.

In the present context by the term “anti-inflammatory compound” is meant a compound which is capable of at least one of the following activities
i) decreasing or inhibiting the gene expression in the immune cells, preferably monocytes/macrophages in response to bacterial products, live bacteria or trauma to produce endogenous inflammatory mediators including receptors for inflammatory mediators and transcription factors involved in the signal transduction of the inflammatory mediators, said mediators being preferably selected from the group comprising cytokines, selected from the group TNFalpha IL-1, IL-6, G-CSF, GM-CSF, M-CSF. Chemokines selected from the group comprising IL-8, MCP-1, receptors selected from the group Tissue factor and IL-2Ralpha,
ii) decrease or inhibit the production bradykinin by the phase contact system,
iii) decrease or inhibit the attractant potential for monocytes, and/or
iv) decrease or inhibit the life-time of monocytes, neutrophils and other immune cells serving as an inducer of apoptosis,
v) decrease or inhibit vascular endothelial cells to express the adhesion molecules, said adhesion molecules being preferably selected from the group comprising PECAM, ICAM-1, E-selectins, VCAM-1
vi) decrease or inhibit activation of the contact phase system to produce bradykinin leading to increased vascular permeability,
vii) stimulate the synthesis of an anti-inflammatory mediator selected from the group of IL-10 and IL-12.
viii) inhibiting complement activation;
ix) decreasing the risk of neural cell degeneration in the presence of chronic neuroinflammation, e.g. neurons which express glutamate N-methyl-D-aspartate receptors.

A peptide sequence of the invention in still another embodiment is capable of modulating cell proliferation, such as stimulating cell proliferation.

Most of the differentiated cell populations in a vertebrate are not permanent: the cells are continually dying and being replaced. New differentiated cell can be produced during adult life in either of two ways: (1) they can form by the simple duplication of the existing differentiated cells, which divide to give pairs of daughter cells of the same type; or (2) they can be generated from stem cells, which are not terminally differentiated (that is, they are not at the end of a pathway of differentiation), can divide without limit (or at least for the lifetime of the animal), and when such cell divide, each daughter has a choice; it can either remain a stem cell or it can embark on a course leading irreversibly to terminal differentiation.

Compounds of the present invention in one preferred embodiment are capable of stimulating cell division, i.e. stimulating proliferation of cells, of both differentiated and stem cells.

Compounds capable of stimulating of terminally differentiated cells are of great value in therapy of traumatised tissues of the body. The peptide sequences of the invention or compounds comprising thereof may for example be used for stimulating proliferation of hepatocytes in case of damage and/or degenerative disease of the liver, or they can be used for stimulating proliferation of endothelial cells, which may be useful in treatment of conditions requiring neoangiogenesis.

Stimulating of stem cell proliferation is another preferred embodiment of use of the peptide sequences of the invention. In a particular preferred embodiment the invention relates to stimulating neural cell precursor proliferation.

Terminally differentiated cells of neural system, in particular neurons, are not capable to proliferate, and therefore compounds that are capable to increase a number of cells in a damaged due trauma or degenerative disease neural cell population are prime targets in a search of new medicine for treatment such conditions. Multiple examples of diseases and pathological conditions where the peptide sequences of the invention may be applied are discussed in the below sections of the description of the further embodiments of the invention.

Testing new compounds for the potential of stimulating cell proliferation may be done as described in Examples below.

### 4. Production of Individual Peptide Sequences

The peptide sequences of the present invention may be prepared by any conventional synthetic methods, recombinant DNA technologies, enzymatic cleavage of full-length proteins which the peptide sequences are derived from, or a combination of said methods.

### Recombinant Preparation

Thus, in one embodiment the peptides of the invention are produced by use of recombinant DNA technologies.

The DNA sequence encoding a peptide or the corresponding full-length protein the peptide originates from may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesised, e.g. in an automatic DNA synthesiser, purified, annealed, ligated and cloned in suitable vectors.

The DNA sequence encoding a peptide may also be prepared by fragmentation of the DNA sequences encoding the corresponding full-length protein of peptide origin, using DNAase I according to a standard protocol (Sambrook et al., Molecular cloning: A Laboratory manual. 2 rd ed., CSHL Press, Cold Spring Harbor, N.Y., 1989). The present invention relates to full-length proteins selected from the groups of proteins identified above. The DNA encoding the full-length proteins of the invention may altematively be fragmented using specific restriction endonucleases. The fragments of DNA are further purified using standard procedures described in Sambrook et al., Molecular cloning: A Laboratory manual. 2 rd ed., CSHL Press, Cold Spring Harbor, N.Y., 1989.

The DNA sequence encoding a full-length protein may also be of genomic or cDNA origin, for instance obtained by preparing a genomic or cDNA library and screening for DNA sequences coding for all or part of the full-length protein by hybridisation using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a peptide or a full-length protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell. Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4-c (Russell et al., 1983, Nature 304:652-654) promoters. Suitable promoters for use in filamentous fungus host cells are, for instance, the ADH3 promoter (McKnight et al., 1985, EMBO J. 4:2093-2099) or the tpiA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or the adenovirus 5 Elb region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op.cit.).

To obtain recombinant peptides of the invention the coding DNA sequences may be usefully fused with a second peptide coding sequence and a protease cleavage site coding sequence, giving a DNA construct encoding the fusion protein, wherein the protease cleavage site coding sequence positioned between the HBP fragment and second peptide coding DNA, inserted into a recombinant expression vector, and expressed in recombinant host cells. In one embodiment, said second peptide selected from, but not limited by the group comprising glutathion-S-reductase, calf thymosin, bacterial thioredoxin or human ubiquitin natural or synthetic variants, or peptides thereof. In another embodiment, a peptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site.

The host cell into which the expression vector is introduced may be any cell which is capable of expression of the peptides or full-length proteins, and is preferably a eukaryotic cell, such as invertebrate (insect) cells or vertebrate cells, e.g. Xenopus laevis oocytes or mammalian cells, in particular insect and mammalian cells. Examples of suitable mammalian cell lines are the HEK293 (ATCC CRL-1573), COS (ATCC CRL-1650), BHK (ATCC CRL-1632, ATCC CCL-10) or CHO (ATCC CCL-61) cell lines. Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al., 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Alternatively, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238-023.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (e.g. in catalogues of the American Type Culture Collection).

The peptides or full-length proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

### Synthetic Preparation

The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

Peptides may for example be synthesised by using Fmoc chemistry and with Acm-protected cysteins. After purification by reversed phase HPLC, peptides may be further processed to obtain for example cyclic or C- or N-terminal modified isoforms. The methods for cyclization and terminal modification are well-known in the art and described in detail in the above-cited manuals.

In a preferred embodiment the peptide sequences of the invention are produced synthetically, in particular, by the Sequence Assisted Peptide Synthesis (SAPS) method.

By SAPS peptides may be synthesised either batchwise in a polyethylene vessel equipped with a polypropylene filter for filtration or in the continuous-flow version of the polyamide solid-phase method (Dryland, A. and Sheppard, R. C., (1986) J. Chem. Soc. Perkin Trans. I, 125-137.) on a fully automated peptide synthesiser using 9-fluorenylmethyloxycarbonyl (Fmoc) or tert.-Butyloxycarbonyl, (Boc) as N-a-amino protecting group and suitable common protection groups for side-chain functionality.

When synthesised, individual peptide sequences may then be formulated as multimers using well-known in the art techniques, for examples dimers of the sequences may be obtained by the LPA method described in WO 00/18791, denrimeric polymers by the MAP synthesis described in PCT/US90/02039.

### 5. Antibody

It is an objective of the present invention to provide an antibody, antigen binding fragment or recombinant protein thereof capable of recognizing and selectively binding to an epitope comprising the motif of the invention or a sequence selected from SEQ ID NOs:1-85, or a fragment of said sequence, preferably the epitope is located on a protein of the S100 family, for example S100A4 or S100A12

By the term “epitope” is meant the specific group of atoms (on an antigen molecule) that is recognized by (that antigen's) antibodies (thereby causing an immune response). The term “epitope” is the equivalent to the term “antigenic determinant”. The epitope may comprise 3 or more amino acid residues, such as for example 4, 5, 6, 7, 8 amino acid residues, located in close proximity, such as within a contiguous amino acid sequence, or located in distant parts of the amino acid sequence of an antigen, but due to protein folding have been approached to each other.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an anti-gen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Novotny J, & Haber E. Proc Natl Acad Sci USA. 82(14):4592-6, 1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and threedimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from anti-body fragments. As used herein, “functional fragment” with respect to antibodies, refers to Fv, F(ab) and F(ab′)₂ fragments.

The term “antibody fragment” is used herein interchangeably with the term “antigen binding fragment”.

Antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it is has similar or immunological properties relative to antibody that binds with specificity to an epitope comprising a peptide sequence selected from any of the sequences identified herein as SEQ ID NOs: 1-85, or a fragment of said sequences. Thus, in context of the present invention the term “antibodv fragment” is identical to term “antigen binding fragment”.

Antibody fragments retain some ability to selectively bind with its antigen or receptor. Some types of antibody fragments are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule can be obtained by treating whole antibody with pepsin, followed by reduction, to yield an intact light chain and a portion of the heavy chain. Two Fab′ fragments are obtained per anti-body molecule.

Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction.
(4) F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.
(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, NY, 1994.

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

The invention contemplate both polyclonal and monoclonal antibody, antigen binding fragments and recombinant proteins thereof which are capable of binding an epitope according to the invention.

The preparation of polyclonal antibodies is well-known to those skilled in the art. See, for example, Green et al. 1992. Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1, which are hereby incorporated by reference.

The preparation of monoclonal antibodies likewise is conventional. See, for example, Kohler & Milstein, Nature, 256:495-7 (1975); Coligan, et al., sections 2.5.1-2.6.7; and Harlow, et al., in: Antibodies: A Laboratory Manual, page 726, Cold Spring Harbor Pub. (1988), Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG). In: Methods in Molecular Biology, 1992, 10:79-104, Humana Press, NY.

Methods of in vitro and in vivo manipulation of monoclonal antibodies are well known to those skilled in the art. For example, the monoclonal antibodies to be used in accordance with the present invention may be made by the hybridoma method first described by Kohler and Milstein, 1975, Nature 256, 495-7, or may be made by recombinant methods, e.g., as described in U.S. Pat. No. 4,816,567. The monoclonal antibodies for use with the present invention may also be isolated from phage antibody libraries using the techniques described in Clackson et al., 1991, Nature 352: 624-628, as well as in Marks et al., 1991, J Mol Biol 222: 581-597. Another method involves humanizing a monoclonal antibody by recombinant means to generate anti-bodies containing human specific and recognizable sequences. See, for review, Holmes, et al., 1997, J Immunol 158:2192-2201 and Vaswani, et al., 1998, Annals Allergy, Asthma & Immunol 81:105-115.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in anti-bodies derived from another species or belonging to another antibody class or subclass, as well as fragments of such antibodies, so long as they exhibit the desired biological activity (U.S. Pat. No. 4,816,567); Morrison et al., 1984, Proc Natl Acad Sci 81: 6851-6855.

Methods of making antibody fragments are also known in the art (see for example, Harlow and Lane, Antibodies: A Laboratory Manual, Cold Spring Harbor Laboratory, NY, 1988, incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain anti-gen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., 1991, In: Methods: A Companion to Methods in Enzymology, 2:97; Bird et al., 1988, Science 242:423-426; U.S. Pat. No. 4,946,778; and Pack, et al., 1993, BioTechnology 11:1271-77.

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain a minimal sequence derived from non-human immunoglobulin, such as the eitope recognising sequence. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity. Humanized antibody(es) containing a minimal sequence(s) of antibody(es) of the invention, such as a sequence(s) recognising the epitope(s) described herein, is one of the preferred embodiments of the invention.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., 1986, Nature 321, 522-525; Reichmann et al., 1988, Nature 332, 323-329; Presta, 1992, Curr Op Struct Biol 2:593-596; Holmes et al., 1997, J Immunol 158:2192-2201 and Vaswani, et al., 1998, Annals Allergy, Asthma & Immunol 81:105-115.

The generation of antibodies may be achieved by any standard method in the art for producing polyclonal and monoclonal antibodies using natural or recombinant fragments of human S100 protein, such as S100A4 or S100A12, said fragment comprising a structural motif of the invention, for example comprising a sequence selected from SEQ ID NOs: 1-85, such as for example a sequences selected from SEQ ID NOs: 1-5, as an antigen. Such antibodies may be also generated using variants, homologues or fragments of peptide sequences of SEQ ID NOs:1-85 said variants, homologues and fragments are immunogenic peptide sequences which meet the following criteria:

(i) being a contiguous amino acid sequence of at least 6 amino acids;
(ii) comprising the motif of the invention.

The antibodies may also be produced in vivo by the individual to be treated, for example, by administering an immunogenic fragment according to the invention to said individual. Accordingly, the present invention further relates to a vaccine comprising an immunogenic fragment described above.

The application also relates to a method for producing an antibody of the invention said method comprising a step of providing of an immunogenic fragment described above.

The invention relates both to antibodies as above, which are capable of modulating, such as enhancing or attenuating, biological function of S100A4 or S100A12, or inhibiting this function. Preferred biological functions of S100A4 and S100A12 in the present context may be a capability of stimulating cell proliferation, cell differentiation or cell survival, promoting nerve regeneration, inhibiting cell migration/dissemination, promoting morphological and functional plasticity, e.g. enhancing synaptic plasticity.

### 6. Pharmaceutical Composition

The invention also relates to a pharmaceutical composition comprising one or more of the compounds defined above, wherein the compound is capable of stimulating neurite outgrowth and/or neural cell differentiation, survival of neural cells and/or stimulating learning and/or memory. Thus, the invention concerns a pharmaceutical composition capable of stimulating differentiation of neuronal cells and/or stimulating regeneration of neuronal cells, and/or stimulating neuronal plasticity in connection with learning and memory, and/or stimulating survival of neural cells.

In the present context the term “pharmaceutical composition” is used synonymously with the term “medicament”.

In a composition the peptide sequences may be formulated as comprising isolated individual peptide fragments or multimers or dimers thereof as discussed above.

The pharmaceutical composition may have the described above effects on cells in vitro or in vivo, wherein the composition is administered to a subject.

The medicament of the invention comprises an effective amount of one or more of the compounds as defined above, or a composition as defined above in combination with the pharmaceutically acceptable additives. Such medicament may suitably be formulated for oral, percutaneous, intramuscular, intravenous, intracranial, intrathecal, intracerebroventricular, intranasal or pulmonal administration.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 5000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. In particular when administering nasally smaller dosages are used than when administering by other routes. Administration may be performed once or may be followed by subsequent administrations. The dosage will also depend on the route of administration and will vary with the age and weight of the subject to be treated. A preferred dosage of multimeric forms would be in the interval 1 mg to 70 mg per 70 kg body weight.

For some indications a localised or substantially localised application is preferred.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to cell death. Preferably the medicament is administered within 8 hours from the factor onset, such as within 5 hours from the factor onset. Many of the cornpounds exhibit a long term effect whereby administration of the compounds may be conducted with long intervals, such as 1 week or 2 weeks.

In connection with the use in nerve guides, the administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

As discussed above, the present invention relates to treatment of individuals for inducing differentiation, stimulating regeneration, plasticity and survival of neural cells in vitro or in vivo, said treatment involving administering an effective amount of one or more compounds as defined above.

Another strategy for administration is to implant or inject cells capable of expressing and secreting the compound in question. Thereby the compound may be produced at the location where it is going to act.

### 7. Treatment

In a further aspect, the present invention relates to said peptides, fragments, or variants thereof for use in the induction of differentiation and/or stimulation of regeneration, plasticity and/or survival of neural cells. The use is for the treatment for preventing diseases and conditions of the central and peripheral nervous system, and of the muscles or of various tissues and organs.

Treatment by the use of the compounds/compositions according to the invention is in one embodiment useful for inducing differentiation, modulating proliferation, stimulate regeneration, neuronal plasticity and survival of cells being implanted or transplanted. This is particularly useful when using compounds having a long term effect.

Thus, the treatment comprises treatment and/or prophylaxis of cell death in relation to diseases or conditions of the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuro-muscular degeneration, schizophrenia, Alzheimer's disease, Parkinson's disease, or Huntington's disease.

Also, in relation to diseases or conditions of the muscles including conditions with impaired function of neuro-muscular connections, such as genetic or traumatic atrophic muscle disorders; or for the treatment of diseases or conditions of various or gans, such as degenerative conditions of the gonads, of the pancreas, such as diabetes mellitus type I and II, of the kidney, such as nephrosis the compounds according to the invention may be used for inducing differentiation, modulating proliferation, stimulate regeneration, neuronal plasticity and survival, i.e. stimulating survival.

In yet a further embodiment the use of the compound and/or pharmaceutical composition is for the stimulation of the ability to learn and/or of the short and/or long term memory.

In particular the compound and/or pharmaceutical composition of the invention may be used in the treatment of clinical conditions, such as psychoses, such as senile and presenile organic psychotic conditions, alcoholic psychoses, drug psychoses, transient organic psychotic conditions, Alzheimer's disease, cerebral lipidoses, epilepsy, general paresis [syphilis], hepatolenticular degeneration, Huntington's chorea, Jakob-Creutzfeldt disease, multiple sclerosis, Pick's disease of the brain, syphilis, Schizophrenic disorders, affective psychoses, neurotic disorders, personality disorders, including character neurosis, nonpsychotic personality disorder associated with organic brain syndromes, paranoid personality disorder, fanatic personality, paranoid personality (disorder), paranoid traits, sexual deviations and disorders, mental retardation, disease in the nerve system and sense organs, cognitive anomalies, inflammatory disease of the central nervous system, such as meningitis, encephalitis, Cerebral degenerations such as Alzheimer's disease, Pick's disease, senile degeneration of brain, communicating hydrocephalus, obstructive hydrocephalus, Parkinson's disease including other extra pyramidal disease and abnormal movement disorders, spinocerebellar disease, cerebellar ataxia, Marie's, Sanger-Brown, Dyssynergia cerebellaris myoclonica, primary cerebellar degeneration, such as spinal muscular atrophy, familial, juvenile, adult spinal muscular atrophy, motor neuron disease, amyotrophic lateral sclerosis, motor neuron disease, progressive bulbar palsy, pseudobulbar palsy, primary lateral sclerosis, other anterior horn cell diseases, anterior horn cell disease, unspecified, other diseases of spinal cord, syringomyelia and syringobulbia, vascular myelopathies, acute infarction of spinal cord (embolic) (nonembolic), arterial thrombosis of spinal cord, edema of spinal cord, subacute necrotic myelopathy, subacute combined degeneration of spinal cord in diseases classified elsewhere, myelopathy, drug-induced, radiationinduced myelitis, disorders of the autonomic nervous system, disorders of peripheral autonomic, sympathetic, parasympathetic, or vegetative system, familial dysautonomia [Riley-Day syndrome], idiopathic peripheral autonomic neuropathy, carotid sinus syncope or syndrome, cervical sympathetic dystrophy or paralysis; peripheral autonomic neuropathy in disorders classified elsewhere, amyloidosis, diseases of the peripheral nerve system, brachial plexus lesions, cervical rib syndrome, costoclavicular syndrome, scalenus anterior syndrome, thoracic outlet syndrome, brachial neuritis or radiculitis, including in newborn. Inflammatory and toxic neuropathy, including acute infective polyneuritis, Guillain-Barre syndrome, Postinfectious polyneuritis, polyneuropathy in collagen vascular disease. Pain syndrome (such as non-opoid pain, neuropatic pain, or in pain related to other disorders, e.g. diabetes or HIV), encephalitis, drug/alcohol abuse, anxiety, perioperative ischemia, psychoses, such as senile and presenile organic psychotic conditions, alcoholic psychoses, drug psychoses, transient organic psychotic conditions, disorders affecting multiple structures of eye, purulent endophthalmitis, diseases of the ear and mastoid process, abnormality of organs and soft tissues in newborn, including in the nerve system, both acute dysfunction and chronic dysfunction (e.g. deficit in cognition mood social functioning) after injury (peripheral and centrally), complications of the administration of anesthetic or other sedation in labor and delivery, diseases in the skin including infection, insufficient circulation problem, injuries, including after surgery, crushing injury, burns. Atrophic dermatitis, psoriasis, infection cased disorders, injuries to nerves and spinal cord, including division of nerve, lesion in continuity (with or without open wound), traumatic neuroma (with or without open wound), traumatic transient paralysis (with or without open wound), accidental puncture or laceration during medical procedure, injury to optic nerve and pathways, optic nerve injury, second cranial nerve, injury to optic chiasm, injury to optic pathways, injury to visual cortex, unspecified blindness, injury to other cranial nerve(s), injury to other and unspecified nerves, poisoning by drugs, medicinal and biological substances, genetic or traumatic atrophic muscle disorders; or for the treatment of diseases or conditions of various organs, such as degenerative conditions of the gonads, of the pancreas, such as diabetes mellitus type I and II, of the kidney, such as nephrosis, metabolic disorders such as obscenity lipid disorders (e.g. hypercholestorolamia, artherslerosis), disorders of endocrine glands, pituitary gland tumor, disorders of amino acid transport and metabolism, disorders of purine and pyrimidine metabolism and gout, bone disorders, such as fracture, osteoporosis, osteo arthritis (OA), stem cell protection or maturation in vivo or in vitro, neurogenesis.

In another embodiment the compound and/or pharmaceutical composition of the invention may be used in the treatment of neoplasms such as malignant neoplasms, benign neoplasms, carcinoma in situ and neoplasms of uncertain behavior, more specifically cancer in breast, thyroidal, pancreas, brain, lung, kidney, prostate, liver, heart, skin, blood organ (incl. but not limited to CML and AML), muscles (sarcoma). Cancers with dysfunction and/or over- or under-expression of specific receptors and/or expression of mutated receptors or associated with soluble receptors, such as but not limited to Erb-receptors and FGF-receptors.

Inflammation of the brain is often consequence of infection, autoimmune processes, toxins, and other conditions. Viral infections are a relatively frequent cause of this condition. Encephalitis may occur as primary or secondary manifestation of TOGAVIRIDAE INFECTIONS; HERPESVIRIDAE INFECTIONS; ADENOVIRIDAE INFECTIONS; FLAVIVIRIDAE INFECTIONS; BUNYAVIRIDAE INFECTIONS; PICORNAVIRIDAE INFECTIONS; PARAMYXOVIRIDAE INFECTIONS; ORTHOMYXOVIRIDAE INFECTIONS; RETROVIRIDAE INFECTIONS; and ARENAVIRIDAE INFECTIONS.

Accordingly, a peptide, compound or a pharmaceutical composition of the invention may be used for treatment inflammation in the brain associated with a viral infection.

A large body of clinical and experimental data indicate that complement activation is an important mechanism for neuronal and glial injury in Guillain-Barré syndromes. Inhibition of complement activation therefore might be expected to limit the progression of the disease (Halstead et al. (2005) Annals of Neurology 58:203-21).

Thus, in another embodiment, a peptide sequence, a compound and pharmaceutical composition may be used for treatment of Guillain-Barré syndrome, its variant forms, such as Miller Fisher syndrome, and other complement dependent neuromuscular disorders.

Peptide sequences, compounds and pharmaceutical composition may also be used for treatment children with autism.

Autism is a brain disorder that begins in early childhood and persists throughout adulthood; affects three crucial areas of development: communication, social interaction, and creative or imaginative play. It is estimated to afflict between 2 and 5 of every 1000 children and is four times more likely to strike boys than girls. Children with autism have difficulties in social interaction and communication and may show repetitive behaviour and have unusual attachments to objects or routines.

In recent years, there have been scientific hints of immune system irregularities in children with autism.

Thus, a peptide sequence, compound or a composition comprising thereof may advantageously be used for treatment inflammation, in particular inflammation of the brain.

A further aspect of the invention is a process of producing a pharmaceutical composition, comprising mixing an effective amount of one or more of the compounds of the invention, or a pharmaceutical composition according to the invention with one or more pharmaceutically acceptable additives or carriers, and administer an effective amount of at least one of said compound, or said pharmaceutical composition to a subject.

In one embodiment of the process as mentioned above, the compounds are used in combination with a prosthetic device, wherein the device is a prosthetic nerve guide. Thus, in a further aspect, the present invention relates to a prosthetic nerve guide, characterised in that it comprises one or more of the compounds or the pharmaceutical composition as defined above. Nerve guides are known in the art.

Another aspect of the invention relates to the use of a compound as defined above. In particular the use of a compound according to the invention is for the production of a pharmaceutical composition. The pharmaceutical composition is preferably for the treatment or prophylaxis of any of the diseases and conditions mentioned above.

In yet a further aspect the invention relates to a method of treating a disease or condition as discussed above by administering a compound as defined herein.

### EXAMPLES

### Peptides

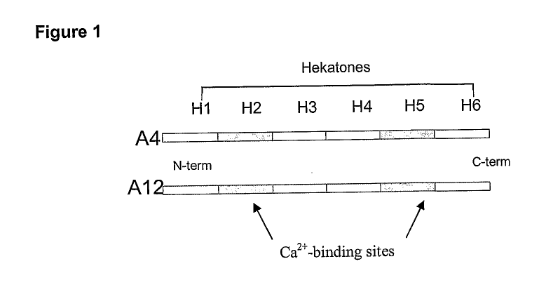

The sequences of S100A4 and S100A12 proteins were divided into six regions each, and the tetrameric peptides representing individual regions were generated. The peptides were termed hekatones (H) 1 to 6 (FIG. 1). Thereafter, all twelve peptides were tested for their neuritogenic activity in primary neurons.

| | | |
|---|---|---|
| S4-3 (A4 H3) | KELLTRELPSFLGKRT | SEQ ID NO: 2 |
| S4-4 (A4 H4) | DEAAFQKLMSNLD | SEQ ID NO: 4 |
| S4-6 (A4 H6) | NEFFEGFPDKQPRKK | SEQ ID NO: 1 |
| S12-1 (A12 H1) | TKLEEHLEGIVNIF | SEQ ID NO: 60 |
| S12-3 (A12 H3) | KQLLTKELANTIKNIK | SEQ ID NO: 3 |
| S12-4 (A12 H4) | DKAVIDEIFQGLD | SEQ ID NO: 33 |
| S12-6 (A12 H6) | HYHTHKE | SEQ ID NO: 75 |

### 1. Primary Neurons: Determination of Neurite Outgrowth

Dissociated hippocampal and cerebellar (CGN) neurons were isolated from Wistar rat embryos at embryonic day 19 or newborn rats as previously described by Rønn et al. (1999). Briefly, hippocampus was isolated from the brain in ice cold modified Krebs Ringer solution, cleared of blood vessels, roughly homogenised by chopping and then trypsinised. The dissociated cells were washed in the presence of DNAse 1 and soybean trypsin inhibitor. Postnatal hippocampal neurons were plated at a density of 10,000 cells/cm² on uncoated 8-well permanox Lab-Tek chamber slides in Neurobasal medium supplemented with 0.4% (w/v) bovine serum albumin (BSA; Sigma-Aldrich), 2% (v/v) B27 Neurobasal supplement, 1% (v/v) glutamax, 100 U/ml penicillin, 100 μg/ml streptomycin and 2% 1 M HEPES (all from Gibco, BRL). After 24 hours, the neurons were fixed with 4% (v/v) formaldehyde for 20 minutes and thereafter immunostained using primary rabbit antibodies against GAP-43 and Alexa Fluor secondary goat anti-rabbit Ig antibodies. Images of at least 200 neurons for each group in each individual experiment were obtained systematically by computer assisted fluorescence microscopy as previously described (Rønn et al., 2000). Briefly, a Nikon Diaphot inverted microscope with a Nikon Plan 20× objective (Nikon, Tokyo, Japan) coupled to a video camera (Grundig Electronics, Germany) was used for recordings. A software package Prima developed at the Protein Laboratory (Copenhagen, Denmark) was used to make a stereologically based determination of neurite length (Rønn et al., 2000).

### Results

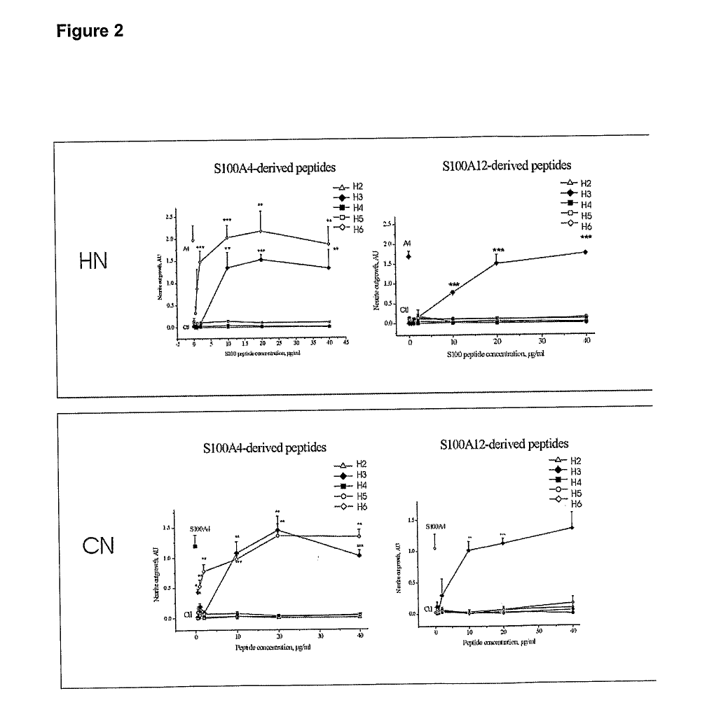

Neuronal cell cultures were treated with various concentrations of a peptide for 24 hours, and the average neurite length was evaluated as described above. It was found that the peptides A4H3, A12H3 and A4H6 strongly induced neurite outgrowth from both cerebellar and hippocampal primary neurons, whereas other peptides derived from the S100A4 and S100A12 proteins had no effect (FIG. 2). Thus peptide segments number 3 and number 6 from various members of the S100 protein family may represent potent pharmacological tools to induce neuronal differentiation and axon regeneration.

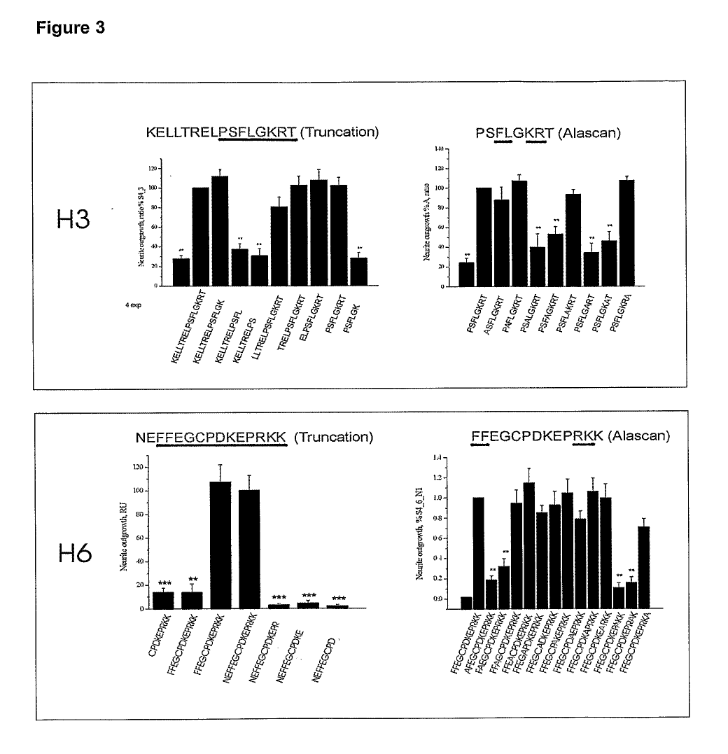

The H3 and H6 peptides derived from S100A4 were further characterized in vitro, and are henceforth referred to as H3 and H6. First, truncated versions of the peptides were tested for their ability to induce neurite outgrowth from primary hippocampal neurons (FIG. 3). Non-truncated versions were used as controls. It was found that only the C-terminal part of the H3 peptide was crucial for the induction of neurite outgrowth, whereas for the H6 peptides truncation of just two amino acids from the N-terminal resulted in the loss of neuritogenic activity. Based on truncation experiments, a minimal sequence required for the induction of neurite outgrowth was identified for each peptide (underlined on top of each truncation graphs). These sequences were subsequently used for the alascan experiments, where individual amino acids were substituted by alanins in order to identify residues crucial for the neurotigenic activities of H3 and H6. We have found four amino acids in each peptide sequence, which were important for the induction of neurite outgrowth of H3 and H6 (underlined on top of respective alascan graphs).

### 2. Activation of Phosphorylated Akt, CREB, and ERK

Primary hippocampal neurons cultured for 6 hours were stimulated with H3 or H6 for 15 minutes. Thereafter the phosphorylation level of the three intracellular messengers involved in neurite outgrowth and survival, Akt, CREB, and ERK, was determined using the PAGE method.

### Results

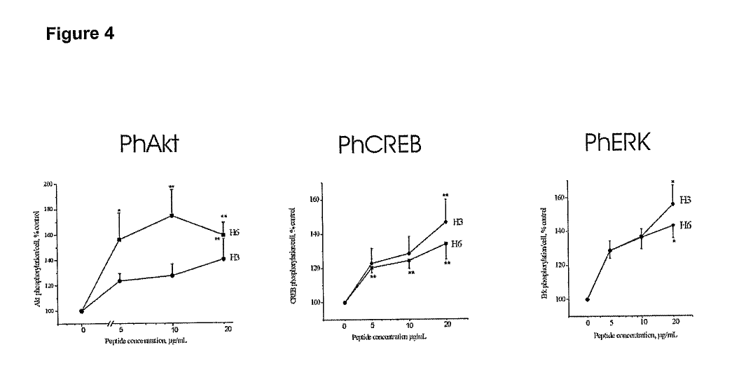

It was found that both peptides significantly increased phosphorylation of all three messengers (FIG. 4).

### 3. Neuronal Survival.

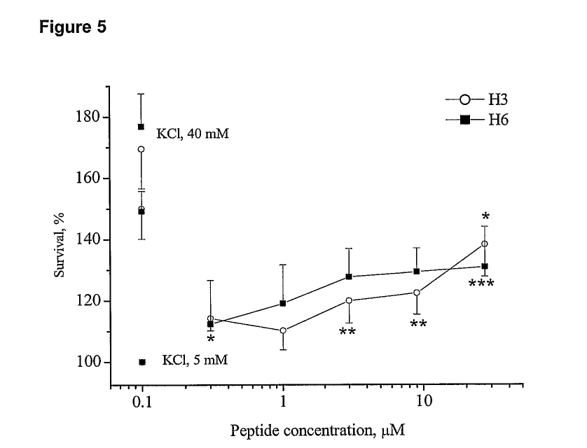

Primary cerebellar neurons were cultured for 7 days in high potassium medium, KCl concentration=40 mM. The primary cerebellar neurons were induced to undergo apoptosis by lowering of the KCl concentration to 5 mM. Both S100A4-derived peptides H3 and H6 partially rescued cerebellar neurons and increased the neuronal survival by 30-40% (FIG. 5).

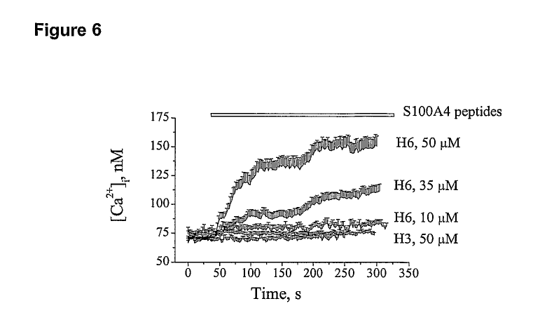

Since Ca²⁺ has been demonstrated to play a crucial role in the processes of neurite outgrowth and survival, and S100A4 is known to increase [Ca²⁺]ᵢ in primary neurons, we loaded cultured hippocampal neurons with a ratiometric Ca²⁺-sensitive dye fura-2 and tested whether the S100A4-derived peptides affected [Ca²⁺]ᵢ. It was found that application of H6, but not H3 significantly increased the level of intracellular Ca²⁺ (FIG. 6).

### 4. Kainic Acid-Induced Toxicity

Male C57BL/6J mice were injected with 10 mg/kg H3 or H6 daily starting from day-2 prior to kainic acid treatment. At day 0 the animals were injected 30 mg/kg KA in order to induce severe seizures, or 20 mg/kg KA in order to induce mild seizures and prevent excessive mortality. Latency and seizure severity were scored for 2 h postinjection by using a numerical scale of 0 to 6: 0—immobility; 1—facial automatism; 2—head nodding; 3—forelimb clonus; 4—rearing; 5—generalized convulsions; 6—death. Control animals received s.c injection of vehicle.

### Results

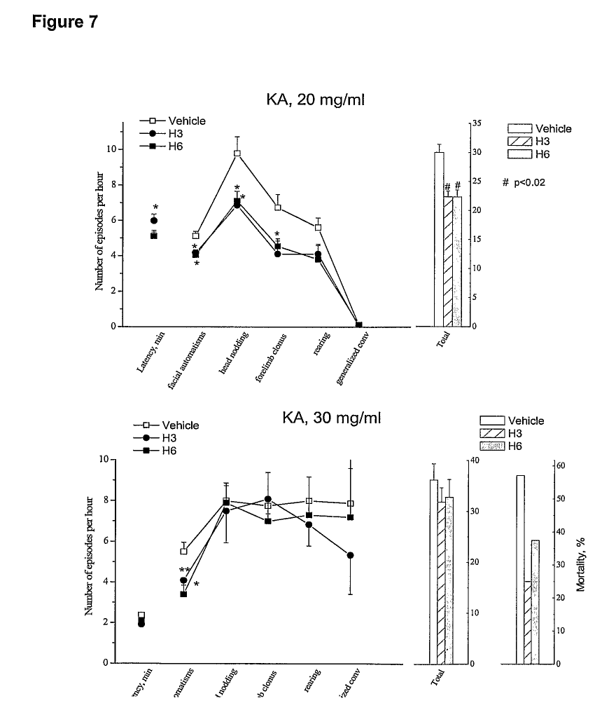

H3 and H6 protect the brain from the kainic acid (KA)-induced toxicity (FIG. 7). Both H3 and H6 decreased the amount of seizures induced by 20 mg/kg KA. Moreover, the peptides shifted the profile of seizures induced by 30 mg/kg KA towards less severe seizures types and decreased the mortality by 30-50%.

5. Functional Recovery after Sciatic Nerve Crush

Nerve crush of sciatic nerve was performed as follows. Before surgery, animals were anesthetized with subcutaneous injection of a Fenthanil/Droperidol/Medazolam (0.8 ml per 100 g). The right hind limb was shaved and swabbed with antiseptic solution. A longitudinal cutaneous incision was made in the back of the thigh. Dissection was carried out along a plane separating the hamstring and gluteal muscles to expose the sciatic nerve. Careful dissection was performed to isolate the sciatic nerve from the surrounding connective tissue over a length of 2 to 2.5 cm. The sciatic nerve was unilaterally crushed twice in the same place (0.5 cm upper trifurcation) for a 30 sec using a locking surgical needle holder. The place of the crush was labelled with 5-0 suture material attached to the epineurium. The wound was closed with 5-0 suture material and rats were allowed to recover. The opposite leg and sciatic nerve were not operated and served as a control. The walking track test was started preoperatively and continuing every second day throughout the duration of the study atsrting at day 6 after the nerve crush. Both hind paws were pressed against a regular stamppad containing water-soluble nontoxic ink, and the rat was allowed to walk freely through a 15×40 cm corridor, toward a darkened box. The bottom of the corridor was lined with paper, upon which the hind pawprints of the rat were recorded. Each rat was allowed to walk 1-3 times per test to obtain the best possible representative tracks. Several prints of each foot were inspected, and the best corresponding pair of prints from experimental (E) and normal (N) legs were measured to the nearest millimeter, using a caliper. Three footprint parameters were measured: distance from the heel to the third toe, print length (PL), the distance between the first and fifth toe, toe spread (TS), and distance between the second and fourth toe, the intermediary toe spread (IT). The three parameters were combined to estimate the Sciatic Functional Index (SFI) as described by Bain et al., 1988. The SFI varies between 0 (for uninjured) and about (−)100 (for maximally impaired gait).

### Results

Wistar rats have been subjected to a sciatic nerve crush at day 0. Thereafter, rats were divided into three groups, each group receiving daily injections of either vehicle (Veh), or the H3 and H6 peptides at a dose of 10 mg/kg for 18 days. Every second day, the Sciatic Function Index (SFI) was evaluated in all groups based on the analysis of walking tracks of individual animals (SFI=−100 corresponding to a cornplete limb paralysis, SFI=0 corresponding to a normal limb function). It has been found that the H3 peptide strongly promoted the recovery of the motor function of the injured limb.

### 6. Traumatic Brain Injury

A focal brain injury on the right fronto-parietal cortex was made by applying a piece of dry-ice (−78° C.) directly onto the skull for 30 seconds in mice and 60 seconds in rats, as previously described in details (Penkowa et al., 2003).

### Tissue Processing

Histochemistry and immunohistochemistry were performed on sections cut from organs taken from fixated animals. Rats and mice were deeply anesthetized with Brietal and flushed by cardiac perfusion with saline containing heparine (0.9% NaCl, 3 ml/L 5000IU heparine) for 2 minutes followed by fixation with Zamboni's fixative (pH 7.4) for 4-8 minutes depending on the size of the animal. For immunohistochemical investigation, brain were dissected and postfixed in Zamboni's for 2-3 hours, dehydraded in graded alcohol followed by xylol and subsequently embedded in paraffin before being cut in 3 μm frontal sections throughout the entire area of the cryo lesion. For heat-induced epitope retrieval, the sections were boiled in citrate buffer (pH 6 or 9) in a microwave oven for 10 minutes followed by incubation in 10% goat serum (In Vitro, Fredensborg, DK) or donkey serum (code BP 005.1; The Binding Site, Birmingham, UK) in tris buffered saline (TBS)/Nonidet P-40 (0.01%) for 30 minutes at room temperature. Sections with mouse tissue used for incubation with monoclonal mouse-derived antibodies were also incubated with Blocking Solutions A+B from the HistoMouse-SP kit to quench endogenous mouse IgG (Zymed, Calif., USA).

### Immunohistochemistry

Following treatment as described above, sections were incubated overnight at 5° C. with one of the following primary antibodies: polyclonal rabbit anti-cow glial fibrillary acidic protein (GFAP) 1:250 (a marker for astrocytes; DakoCytomation); monoclonal mouse anti-rat proliferating cellular nuclear antigen (PCNA) 1:10 (marking proliferating cell nuclear antigen/cell proliferation; DakoCytomation). The primary antibody was detected with biotynilated secondary antibodies, incubated with streptavidinbiotin-peroxidase complex for 30 min.

### In Situ Detection of DNA Fragmentation (TUNEL)

Terminal deoxynucleotidyl transferase (TdT)-mediated deoxyuridine triphosphate (dUTP)-biotin nick end labelling (TUNEL) was performed using the Fragment End Labeling (FragEL™) Detection Kit (Calbiochem, USA, code QIA33). The FragEL kit contains all the materials used below and each step was performed according to the manufacturer's recommendations. The tissue was processed and rehydrated as mentioned above, and sections were incubated with 20 □g/ml proteinase K for 20 min to strip off nuclear proteins. After immersion in equilibration buffer for 20 min, sections were incubated with TdT and biotin-labeled deoxynucleotids (dNTP-biotin) in a humified chamber at 37° C. for 1.5 hr. This was followed by wash buffer and the stop solution for 5 min at room temperature to stop the reaction. After washing in TBS and incubation in blocking buffer for 10 min, the sections were incubated with Peroxidase-Streptavidin for 30 min and afterwards, DAB was used as chromogen. The sections were counterstained with methyl-green. Negative control sections were treated similarly but incubated in the absence of TdT enzyme or dNTP-biotin or Peroxidase-Streptavidin. We also compared our sections with positive control slides provided in the FragEL Detection Kit. Furthermore, TUNEL was compared with other stainings for apoptosis (activated caspase-3, cytochrome-c and TNF□-R1). In addition, the morphologic criteria for apoptosis, i.e. cell shrinkage, formation of apoptotic bodies, membrane blebbing, no loss of cellular integrity, compaction of chromatin into uniformly dense masses, were evaluated.

### Cell Counts and Statistical Analysis

In addition to morphological evaluation, quantification (cellular counts) of some of the variables, i.e. GFAP-positive cells, the area of GFAP-immunoreactivity, % of TUNEL-positive cells, % of proliferating cells, were analyzed. The analysis was carried out from the penumbra area of 3 μm brain sections for statistical analysis of the results. For each parameter analysed, cell counts were performed in at least 2 sections from each brain and from at least 5 animals from each group and a mean value was calculated. To this end, positively stained cells were defined as cells with staining of the soma. However, in the case of apoptotic markers (like TUNEL) positively stained cells were defined as cells with nuclear staining. The cell counts were performed by the same investigator, who was blinded to animal identity and the different treatments. Cells were counted at the border of the cortical lesion (the rim between lesioned and unlesioned brain tissue), where inflammation is prominent. Results obtained from sectioned brains were analysed with two-way ANOVA where applicable (as judged by Levine's test) or the Mann-Whitney test. Results obtained from cell cultures were analyzed with student's t-test.

### Results

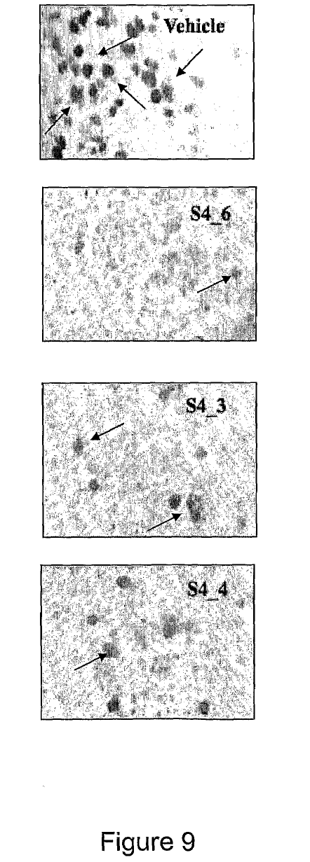

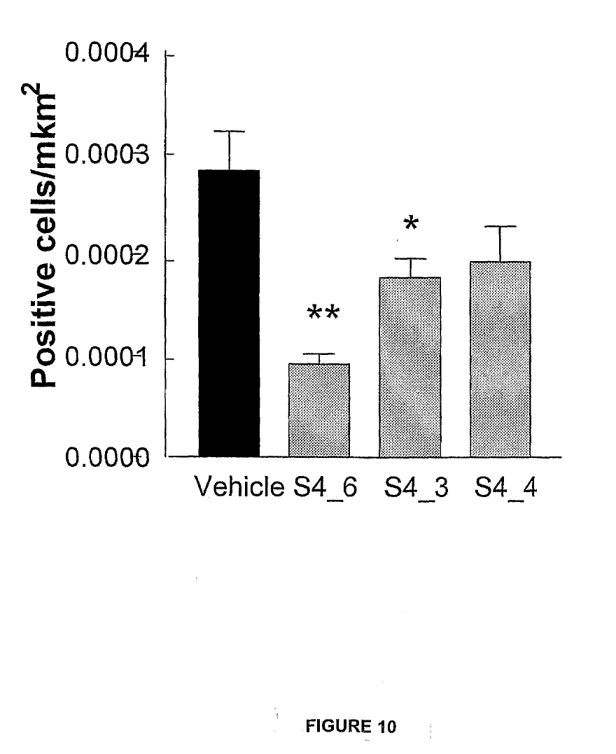

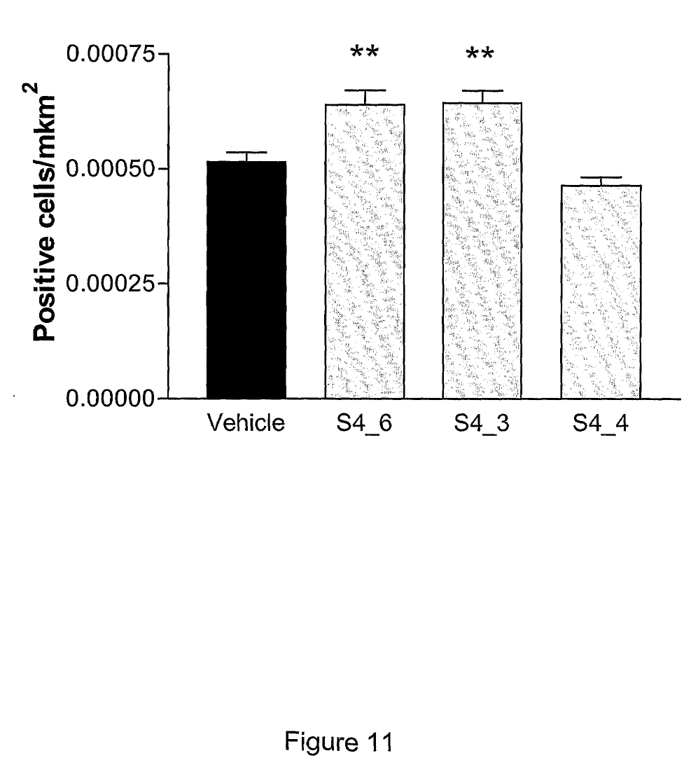

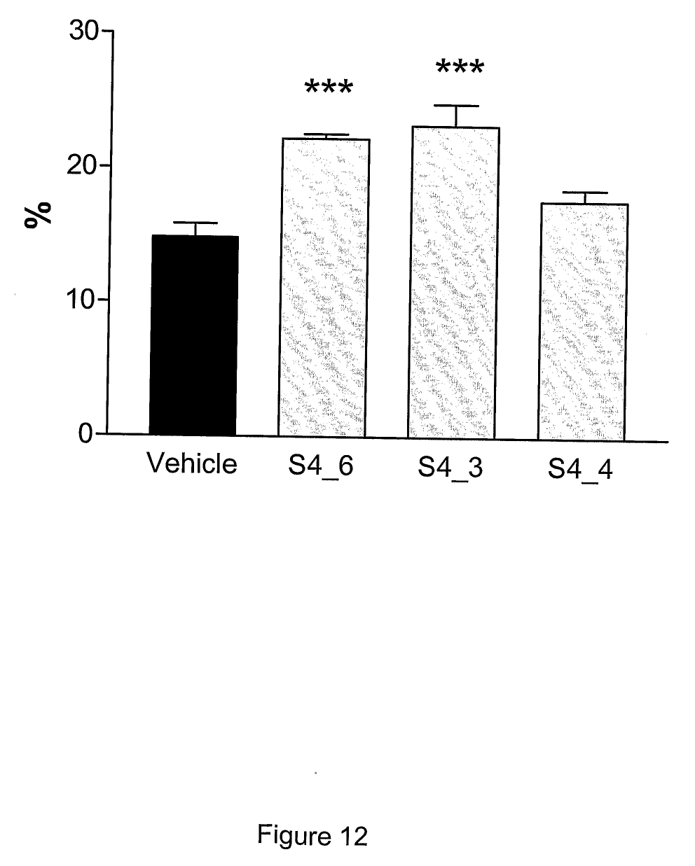

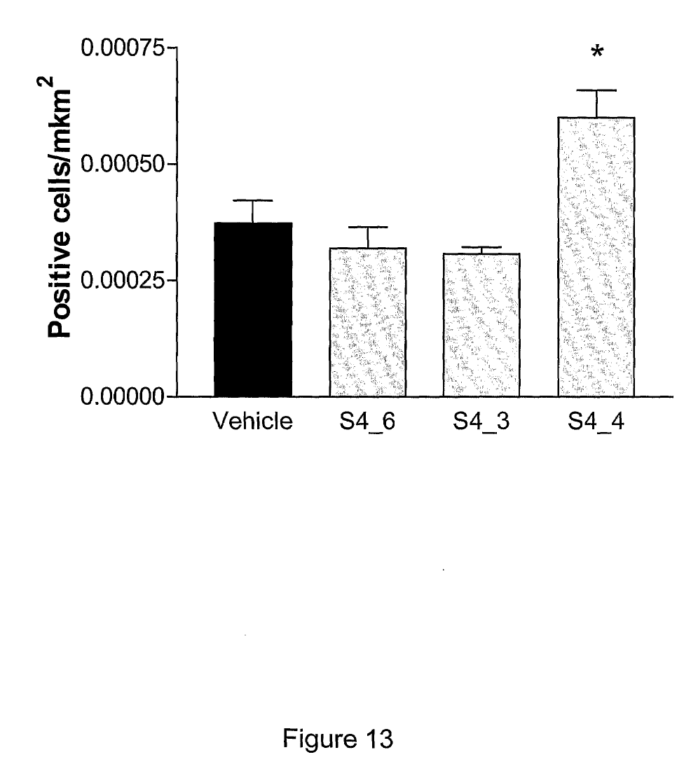

(FIGS. 9, 10, 11, 12, 13). Treatment with the peptides (10 mg/kg/administration) was started one day before the lesion and continued on the next day and the third day after the lesion. The animals were perfused on the fourth day after the lesion. It was found that both the S4-3 and S4-6 peptides protected cells in the cortex from cell death induced by cryolesion to undergo apoptosis. Treatment with each of the two peptides induced astrogliosis as reflected by an increase of the number of GFAP-positive cells and in the area of GFAP-immunoreactivity. On the other hand, the A4H4 peptide strongly induced proliferation of neural progenitor cells.

### CONCLUSION

The peptides S4-3 (A4H3), S4-6 (A4H6) and S12-6 (A12H6) are potent stimulators of neurite outgrowth in vitro.

The peptides S4-3 (A4H3) and S4-6 (A4H6) are neuroprotective in vivo.

The peptide S4-4 (A4H4) has the potential to induce neurogenesis in animals after brain injury.

1. A peptide comprising at most 30 contiguous amino acid residues comprising an amino acid motif of the formula:
x^{p1}-(x1)-(x2)-(x3)-x^{p2}-(x4),
wherein
x^{p1} and x^{p2} are hydrophobic amino acid residues,
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

2. The peptide according to claim 1, wherein the motif comprises a threeamino acid residues sequence wherein two amino acid residues are hydrophobic residues contiguous to each other and the third amino acid residue is a residue selected from E, D, K, R, Q, N, S or T.

3. The peptide according to claim 2, wherein the sequence comprises residues (x3), x^{p2} and (x4), wherein
(x3) is an amino acid residue selected from E, D, K, R, Q, N, S or T and (x4) is a hydrophobic amino acid residue, or wherein
(x4) is an amino acid residue selected from E, D, K, R, Q, N, S or T and (x3) residue is a hydrophobic amino acid residue.

4. The peptide according to claim 1, wherein the peptide comprises 6 to 25 contiguous amino acid residues.

5. The peptide according to claim 1, wherein the peptide comprises 6 to 15 contiguous amino acid residues.

6. The peptide according to claim 1, wherein the peptide is capable of stimulating cell survival, cell proliferation and/or cell differentiation.

7. The peptide according to claim 6, wherein the cell is a cell of the neural system.

8. The peptide according to claim 6, wherein the cell is a neuronal cell.

9. The peptide according to claim 1, wherein the peptide is capable of stimulating neural plasticity associated with memory and learning.

10. The peptide according to claim 1, wherein the peptide comprises an amino acid sequence selected from SEQ ID NOs:1-85, or a fragment or a variant of said sequence.

11. The peptide according to claim 10, wherein the fragment is an amino acid sequence comprising at least 5 amino acid residues comprising two contiguous hydrophobic amino acid residues and a residue selected from K, R, D, E, N, Q, S or T which follows or precedes said hydrophobic residues.

12. The peptide according to claim 10, wherein the variant is an amino acid sequence of at least 6 amino acid residues having at least 50% sequence similarity with a sequence selected from the sequences of SEQ ID NOs: 1-85.

13. The peptide according to claim 10, wherein the peptide is derived from the family of S100 proteins.

14. The peptide according to claim 13, wherein the peptide has a sequence selected from SEQ ID NOs:1-85.

15. The peptide according to claim 13, wherein the peptide has a sequence selected from SEQ ID NOs: 1-5.

16. The peptide according to claim 13, wherein the peptide has a sequence selected from SEQ ID NOs: 51-68.

17. The peptide according to claim 13, wherein the peptide has a sequence selected from SEQ ID NOs:6-22.

18. The peptide according to claim 13, wherein the peptide has a sequence selected from SEQ ID NOs:23-42.

19. The peptide according to claim 12, wherein the peptide has a sequence selected from SEQ ID NOs:43-50.

20. The peptide according to claim 12, wherein the peptide has a sequence selected from SEQ ID NOs:52-68.

21. The peptide according to claim 13, wherein the peptide has a sequence selected from SEQ ID NOs:69-85.

22. A compound comprising a peptide of claim 1.

23. The compound according to claim 22, wherein the peptide is formulated as monomer consisting of the single copy of an individual peptide sequence.

24. The compound according to claim 22, wherein the peptide is formulated as a multimer consisting of two or more copies of an individual peptide sequence, such as a dimer or tetramer of an individual peptide sequence.

25. The compound of claim 24, wherein the multimer is a dendrimer.

26. (canceled)

27. A pharmaceutical composition comprising a peptide according to claim 1.

28. A method of stimulating neural cell survival, differentiation, proliferation and/or plasticity associated with memory and learning comprising:
using a peptide, compound or pharmaceutical composition comprising an amino acid motif of the formula:
x^{p1}-(x1)-(x2)-(x3)-x^{p2}-(x4), wherein x^{p1} and x^{p2} are hydrophobic amino acid residues,
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

29. A method of treatment of a condition or disease wherein stimulating neural cell survival, differentiation, proliferation and/or plasticity, such as plasticity associated with learning and memory is beneficial for treatment, comprising:
administering an effective amount of a peptide, compound or pharmaceutical composition comprising an amino acid motif of the formula:
x^{p1}-(x1)-(x2)-(x3)-x^{p2}-(x4), wherein x^{p1} and x^{p2} are hydrophobic amino acid residues,
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

30. The method according to claim 29, wherein the condition or disease is a condition or disease of the central and peripheral nervous system.

31. The method according to claim 30, wherein the condition or disease is selected from postoperative nerve damage, traumatic nerve damage, impaired myelination of nerve fibers, postischaemic damage, such as after stroke, nerve degeneration associated with diabetes mellitus, disorders affecting the circadian clock or neuromuscular transmission.

32. The method according to claim 29, wherein the condition or disease is selected of conditions or diseases of the muscles including conditions with impaired function of neuro-muscular connections, such as after organ transplantation, or such as genetic or traumatic atrophic muscle disorders.

33. The method according to claim 29, wherein the condition or disease is an impaired ability to learn and/or impaired memory.

34. The method according to claim 29, wherein the condition or disease is Parkinson's disease, Alzheimer's disease, Huntington's disease or dementia such as multiinfarct dementia.

35. The method according to claim 29, wherein the condition or disease is a mental disease, such as a disorder of thought and/or mood, neuropsychiatric disorders including bipolar (BPD), genetically related unipolar affective disorders, delusional disorders, paraphrenia, paranoid psychosis, schizophrenia, schizotypal disorder, schizoaffective disorder, schizoaffective bipolar and genetically related unipolar affective disorders, psychogenic psychosis, catatonia, periodic bipolar and genetically related unipolar affective disorders, cycloid psychosis, schizoid personality disorder, paranoid personality disorder, bipolar and genetically related unipolar affective disorders related affective disorders and subtypes of unipolar affective disorder.

36. The method according to claim 29, wherein the disease is prion disease.

37. The method according to claim 29, wherein the condition or disease is Guillain-Barré syndrome, its variant form, such as Miller Fisher syndrome, or another complement dependent neuromuscular disorder.

38. A method of treating cancer, comprising:
administering an effective amount of a peptide, compound or pharmaceutical composition comprising an amino acid motif of the formula:
x^{p1}-(x1)-(x2)-(x3)-x^{p2}-(x4), wherein x^{p1} and x^{p2} are hydrophobic amino acid residues,
at least one of (x1), (x2) or (x3) is an amino acid residue selected from E, D, K, R, Q, N, S or T and
(x4) is an amino acid residue selected from E, D, K, R, Q, N, S or T or a hydrophobic amino acid residue.

39. The method according to claim 38, wherein the cancer is any cancer involving neoangiogenesis.

40. The method according to claim 38, wherein the cancer is a cancer of neural system.

41. A method of treating body damages due to alcohol consumption, comprising:
administering an effective amount of a peptide, compound or pharmaceutical composition comprising an amino acid motif of the formula:
x^{p1}-(x1)-(x2)-(x3)-x^{p2}-(x4), wherein x^{p1} and x^{p2} are hydrophobic amino acid residues,
at least one of (x1), (x2) or (x3) is an amino acid residue selected from E, D, K, R, Q, N, S or T, and
(x4) is an amino acid residue selected from E, D, K, R, O, N, S or T or a hydrophobic amino acid residue.

42. A method of treating a condition or disease which is characterized by the presence of a sustained inflammatory response, comprising:
administering an effective amount of a peptide, compound or pharmaceutical composition comprising an amino acid motif of the formula:
x^{p1}-(x1)-(x2)-(x3)-x^{p2}-(x4), wherein x^{p1} and x^{p2} are hydrophobic amino acid residues,
at least one of (x1), (x2) or (x3) is an amino acid residue selected from E, D, K, R, Q, N, S or T, and
(x4) is an amino acid residue selected from E, D, K, R, O, N, S or T or a hydrophobic amino acid residue.

43. The method according to claim 42, wherein the condition or disease is brain inflammation or autoimmune disease.

44. (canceled)

45. An antibody capable of recognizing and binding to an epitope comprising the amino acid motif according to claim 1.

46. The antibody according to claim 45, wherein the epitope comprises an amino acid sequence selected from SEQ ID NOs:1-85, or a fragment or variant of said sequence.

47. A method of treating a condition or disease, comprising:
administering to an individual in need an effective amount of an antibody according to claim 45.

48. A pharmaceutical composition comprising compound according to claim 22.
